(19) United States
(12) Patent Application Publication
(10) Pub. No.: US 2013/0102618 A1
(43) Pub. Date: Apr. 25, 2013

(54) 3-AMINO-5,6-DIHYDRO-1H-PYRAZIN-2-ONE DERIVATIVES USEFUL FOR THE TREATMENT OF ALZHEIMER'S DISEASE AND OTHER FORMS OF DEMENTIA

(71) Applicants: Francisca Delgado-Jiménez, Toledo (ES); Gary John Tresadern, Toledo (ES); Andrés Avelino Trabanco-Suárez, Toledo (ES)

(72) Inventors: Francisca Delgado-Jiménez, Toledo (ES); Gary John Tresadern, Toledo (ES); Andrés Avelino Trabanco-Suárez, Toledo (ES)

(73) Assignee: JANSSEN PHARMACEUTICA NV, New Brunswick, NJ (US)

(21) Appl. No.: 13/806,214

(22) PCT Filed: Jun. 27, 2011
(86) PCT No.: PCT/EP2011/060712

§ 371 Date: Dec. 21, 2012

(30) Foreign Application Priority Data
Jun. 28, 2010 (EP) 10167542.9

(51) Int. Cl. C07D 241/08; C07D 401/10; C07D 403/10

(52) U.S. Cl. CPC C07D 241/08; C07D 403/10; C07D 401/10
USPC 514/255.05; 514/255.06; 544/295; 544/405; 544/408

(57) ABSTRACT

The present invention relates to novel 3-amino-5,6-dihydro-1H-pyrazin-2-one derivatives as inhibitors of beta-secretase, also known as beta-site amyloid cleaving enzyme, BACE, BACE1, Asp2, or memapsin2. The invention is also directed to pharmaceutical compositions comprising such compounds, to processes for preparing such compounds and compositions, and to the use of such compounds and compositions for the prevention and treatment of disorders in which beta-secretase is involved, such as Alzheimer's disease (AD), mild cognitive impairment, senility, dementia, dementia with Lewy bodies, Down's syndrome, dementia associated with stroke, dementia associated with Parkinson's disease or dementia associated with beta-amyloid.

### FIELD OF THE INVENTION

The present invention relates to novel 3-amino-5,6-dihydro-1H-pyrazin-2-one derivatives as inhibitors of beta-secretase, also known as beta-site amyloid cleaving enzyme, BACE, BACE1, Asp2, or memapsin2. The invention is also directed to pharmaceutical compositions comprising such compounds, to processes for preparing such compounds and compositions, and to the use of such compounds and compositions for the prevention and treatment of disorders in which beta-secretase is involved, such as Alzheimer's disease (AD), mild cognitive impairment, senility, dementia, dementia with Lewy bodies, Down's syndrome, dementia associated with stroke, dementia associated with Parkinson's disease or dementia associated with beta-amyloid.

### BACKGROUND OF THE INVENTION

Alzheimer's Disease (AD) is a neurodegenerative disease associated with aging. AD patients suffer from cognition deficits and memory loss as well as behavioral problems such as anxiety. Over 90% of those afflicted with AD have a sporadic form of the disorder while less than 10% of the cases are familial or hereditary. In the United States, about 1 in 10 people at age 65 have AD while at age 85, 1 out of every two individuals are affected with AD. The average life expectancy from the initial diagnosis is 7-10 years, and AD patients require extensive care either in an assisted living facility which is very costly or by family members. With the increasing number of elderly in the population, AD is a growing medical concern. Currently available therapies for AD merely treat the symptoms of the disease and include acetylcholinesterase inhibitors to improve cognitive properties as well as anxiolytics and antipsychotics to control the behavioral problems associated with this ailment.

The hallmark pathological features in the brain of AD patients are neurofibillary tangles which are generated by hyperphosphorylation of tau protein and amyloid plaques which form by aggregation of beta-amyloid 1-42 (Abeta 1-42) peptide. Abeta 1-42 forms oligomers and then fibrils, and ultimately amyloid plaques. The oligomers and fibrils are believed to be especially neurotoxic and may cause most of the neurological damage associated with AD. Agents that prevent the formation of Abeta 1-42 have the potential to be disease-modifying agents for the treatment of AD. Abeta 1-42 is generated from the amyloid precursor protein (APP), comprised of 770 amino acids. The N-terminus of Abeta 1-42 is cleaved by beta-secretase (BACE), and then gamma-secretase cleaves the C-terminal end. In addition to Abeta 1-42, gamma-secretase also liberates Abeta 1-40 which is the predominant cleavage product as well as Abeta 1-38 and Abeta 1-43. These Abeta forms can also aggregate to form oligomers and fibrils. Thus, inhibitors of BACE would be expected to prevent the formation of Abeta 1-42 as well as Abeta 1-40, Abeta 1-38 and Abeta 1-43 and would be potential therapeutic agents in the treatment of AD.

### SUMMARY OF THE INVENTION

The present invention is directed to a compound of Formula (I)

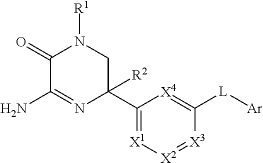

or a stereoisomeric form thereof, wherein
R¹ is selected from the group consisting of hydrogen, C₁₋₃alkyl, mono- and polyhalo-C₁₋₃alkyl, aryl and heteroaryl;
R² is selected from the group consisting of hydrogen, C₁₋₃alkyl, mono- and polyhalo-C₁₋₃alkyl, aryl and heteroaryl;
X¹, X², X³, X⁴ are independently C(R³) or N, provided that no more than two thereof represent N; each R³ is selected from the group consisting of hydrogen, halo, C₁₋₃ alkyl, mono- and polyhalo-C₁₋₃alkyl, cyano, C₁₋₃alkyloxy, mono- and polyhalo-C₁₋₃alkyloxy;
L is a bond or —N(R⁴)CO—, wherein R⁴ is hydrogen or C₁₋₃alkyl;
Ar is homoaryl or heteroaryl;
wherein homoaryl is phenyl or phenyl substituted with one, two or three substituents selected from the group consisting of halo, cyano, C₁₋₃alkyl, C₁₋₃alkyloxy, mono- and polyhalo-C₁₋₃alkyl;
heteroaryl is selected from the group consisting of pyridyl, pyrimidyl, pyrazyl, pyridazyl, furanyl, thienyl, pyrrolyl, pyrazolyl, imidazolyl, triazolyl, thiazolyl, thiadiazolyl, oxazolyl, and oxadiazolyl, each optionally substituted with one, two or three substituents selected from the group consisting of halo, cyano, C₁₋₃ alkyl, C₁₋₃alkyloxy, mono- and polyhalo-C₁₋₃alkyl; or
an addition salt or a solvate thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of treating a disorder mediated by the beta-secretase enzyme, comprising administering to a subject in need thereof a therapeutically effective amount of any of the compounds or pharmaceutical compositions described above.

Further exemplifying the invention are methods of inhibiting the beta-secretase enzyme, comprising administering to a subject in need thereof a therapeutically effective amount of any of the compounds or pharmaceutical compositions described above.

An example of the invention is a method of treating a disorder selected from the group consisting of Alzheimer's disease, mild cognitive impairment, senility, dementia, dementia with Lewy bodies, Down's syndrome, dementia associated with stroke, dementia associated with Parkinson's disease and dementia associated with beta-amyloid, preferably Alzheimer's disease, comprising administering to a subject in need thereof, a therapeutically effective amount of any of the compounds or pharmaceutical compositions described above.

Another example of the invention is any of the compounds described above for use in treating: (a) Alzheimer's Disease, (b) mild cognitive impairment, (c) senility, (d) dementia, (e) dementia with Lewy bodies, (f) Down's syndrome, (g) dementia associated with stroke, (h) dementia associated with Parkinson's disease and (i) dementia associated with beta-amyloid, in a subject in need thereof.

### DETAILED DESCRIPTION OF THE INVENTION

The present invention is directed to compounds of formula (I) as defined hereinbefore, and pharmaceutically acceptable salts thereof. The compounds of formula (I) are inhibitors of the beta-secretase enzyme (also known as beta-site cleaving enzyme, BACE, BACE1, Asp2 or memapsin 2), and are useful in the treatment of Alzheimer's disease, mild cognitive impairment, senility, dementia, dementia associated with stroke, dementia with Lewy bodies, Down's syndrome, dementia associated with Parkinson's disease and dementia associated with beta-amyloid, preferably Alzheimer's disease, mild cognitive impairment or dementia, more preferably Alzheimer's disease.

In an embodiment of the present invention, R¹ and R² are independently selected from C₁₋₃ alkyl;

X¹, X², X³, X⁴ are independently C(R³) wherein each R³ is selected from hydrogen and halo;
L is a bond or —N(R⁴)CO—, wherein R⁴ is hydrogen;
Ar is homoaryl or heteroaryl;
wherein homoaryl is phenyl or phenyl substituted with one or two substituents selected from the group consisting of halo, cyano, C₁₋₃alkyl, and C₁₋₃alkyloxy;
heteroaryl is selected from the group consisting of pyridyl, pyrimidyl, and pyrazyl, each optionally substituted with one or two substituents selected from the group consisting of halo, cyano, C₁₋₃ alkyl, and C₁₋₃alkyloxy; or
an addition salt or a solvate thereof.

In another embodiment of the present invention, R¹ and R² are methyl;

### X¹, X², X³, X⁴ are CH;

L is a bond or —N(R⁴)CO—, wherein R⁴ is hydrogen;
Ar is homoaryl or heteroaryl;
wherein homoaryl is phenyl or phenyl substituted with one or two substituents selected from chloro and cyano;
heteroaryl is selected from the group consisting of pyridyl, pyrimidyl, and pyrazyl, each optionally substituted with one or two substituents selected from the group consisting of chloro, fluoro, cyano, methyl, and methoxy; or
an addition salt or a solvate thereof.

In another embodiment of the present invention R¹ and R² are methyl;

### X¹ is CH or CF; X², X³ and X⁴ are CH;

### L is —NHCO—;

Ar is 5-chloro-pyridin-2-yl; or
an addition salt or a solvate thereof.

In another embodiment of the present invention R¹ and R² are methyl;

### X¹ and X³ are CH or CF; X² and X⁴ are CH;

L is a bond;
Ar is 5-methoxy-pyridin-3-yl or pyrimidin-5-yl; or
an addition salt or a solvate thereof.

### DEFINITIONS

“Halo” shall denote fluoro, chloro and bromo; “C₁₋₃alkyl” shall denote a straight or branched saturated alkyl group having 1, 2 or 3 carbon atoms, e.g. methyl, ethyl, 1-propyl and 2-propyl; “C₁₋₃alkyloxy” shall denote an ether radical wherein C₁₋₃alkyl is as defined before; “mono- and polyhaloC₁₋₃alkyl” shall denote C₁₋₃alkyl as defined before, substituted with 1, 2, 3 or where possible with more halo atoms as defined before; “mono- and polyhaloC₁₋₃alkyloxy” shall denote an ether radical wherein mono- and polyhaloC₁₋₃alkyl is as defined before; “C₃₋₆cycloalkyl” shall denote cyclopropyl, cyclobutyl, cyclopentyl and cyclohexyl; “C₃₋₆cycloalkanediyl” shall denote a bivalent radical such as cyclopropanediyl, cyclobutanediyl, cyclopentanediyl and cyclohexane-diyl.

The term “subject” as used herein, refers to an animal, preferably a mammal, most preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

It will be appreciated that some of the compounds according to formula (I) and the addition salts, hydrates and solvates thereof may contain one or more centers of chirality and exist as stereoisomeric forms.

Hereinbefore and hereinafter, the term “compound of formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

For use in medicine, the salts of the compounds of this invention refer to non-toxic “pharmaceutically acceptable salts”. Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable salts include, but are not limited to, the following: acetic acid, 2,2-dichloro-acetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxyethane-sulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable salts include, but are not limited to, the following: ammonia, L-arginine, benethamine, benzathine, calcium hydroxide, choline, dimethylethanolamine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The chemical names of the compounds of the present invention were generated according to the nomenclature rules agreed upon by the Chemical Abstracts Service. Some of the compounds according to formula (I) may also exist in their tautomeric form. Such forms although not explicitly indicated in the above formula are intended to be included within the scope of the present invention.

### Preparation of the Compounds

### A. Preparation of the Final Compounds

### Experimental Procedure 1

The final compounds according to Formula (I-a) wherein L is —N(R⁴)CO—, can be prepared by reacting an intermediate compound of Formula (II-a) with a compound of Formula (III) according to reaction scheme (1), a reaction that is performed in a suitable reaction-inert solvent, such as, for example, N,N-dimethylformamide, in the presence of a suitable base, such as, for example, K₃PO₄, a copper catalyst such as, for example, CuI and a diamine such as for example (1R,2R)-(−)-1,2-diaminocyclohexane, under thermal conditions such as, for example, heating the reaction mixture at 180° C., for example for 135 minutes under microwave irradiation. In reaction scheme (1), all variables are defined as in Formula (I) and W is halo.

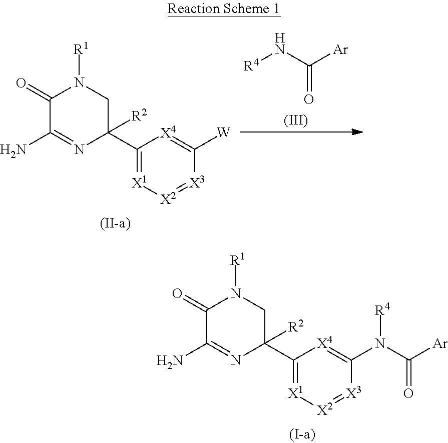

### Experimental Procedure 2

Additionally, the final compounds according to Formula (I-a), can be prepared by reacting an intermediate compound of Formula (II-b) with a compound of Formula (IV) according to reaction scheme (2), a reaction that is performed in a suitable reaction-inert solvent, such as, for example, dichloromethane, in the presence of a suitable base, such as, for example, triethylamine, in the presence of a condensation agent such as for example O-(7-azabenzotriazol-1-yl)-N,N,N′,N′-tetramethyluronium hexafluorophosphate [HATU, CAS 148893-10-1], under thermal conditions such as, for example, heating the reaction mixture at 25° C., for example for 2 hours. In reaction scheme (2), all variables are defined as in Formula (I).

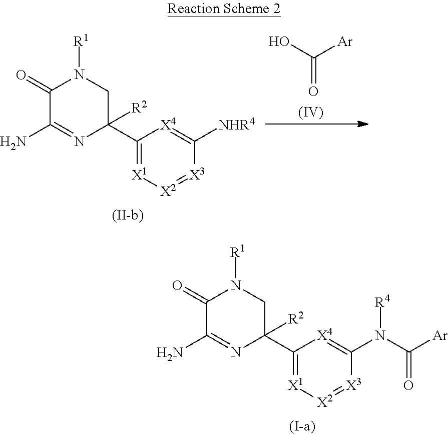

### Experimental Procedure 3

Additionally, the final compounds according to Formula (I-a), can be prepared by reacting an intermediate compound of Formula (II-b) with a compound of Formula (V) according to reaction scheme (3), a reaction that is performed in a suitable reaction-inert solvent, such as, for example, dichloromethane, in the presence of a suitable base, such as, for example, pyridine, under thermal conditions such as, for example, heating the reaction mixture at 25° C., for example for 2 hours. In reaction scheme (3), all variables are defined as in Formula (I) and Y is halo.

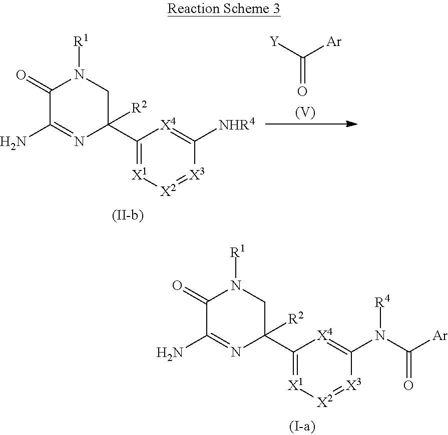

### Experimental Procedure 4

The final compounds according to Formula (I-b) wherein L is a bond, can be prepared by reacting an intermediate compound of Formula (II-a) with a compound of Formula (VI) according to reaction scheme (4), a reaction that is performed in a suitable reaction-inert solvent, such as, for example, mixtures of inert solvents such as, for example, 1,4-dioxane/ethanol, in the presence of a suitable base, such as, for example, aqueous K₃CO₃, a Pd-complex catalyst such as, for example, tetrakis-(triphenylphosphine)palladium (0) [CAS 14221-01-3] under thermal conditions such as, for example, heating the reaction mixture at 80° C., for example for 20 hours or for example, heating the reaction mixture at 150° C., for 10 minutes to 30 minutes under microwave irradiation. In reaction scheme (4), all variables are defined as in Formula (I) and W is, halo. R⁵ and R⁶ may be hydrogen or alkyl, or may be taken together to form for example a bivalent radical of formula —CH₂CH₂—, —CH₂CH₂CH₂—, or —C(CH₃)₂C(CH₃)₂—.

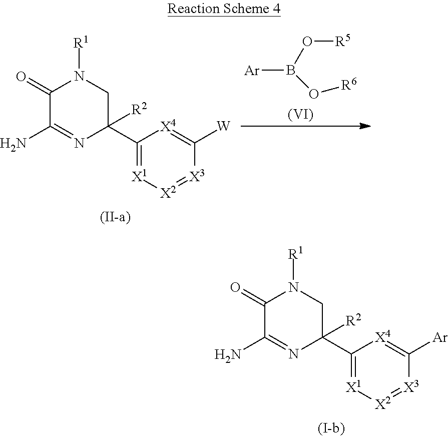

A number of intermediates and starting materials in the foregoing preparations are known compounds which may be prepared according to art-known methodologies of preparing said or similar compounds and some intermediates are new. A number of such preparation methods will be described hereinafter in more detail.

### B. Preparation of the Intermediate Compounds

### Experimental Procedure 5

The intermediate compounds of Formula (II-a), (II-b) and (II-c), wherein R¹ and R² are hydrogen can generally be prepared following the reaction steps shown in the reaction schemes (5) and (6) below.

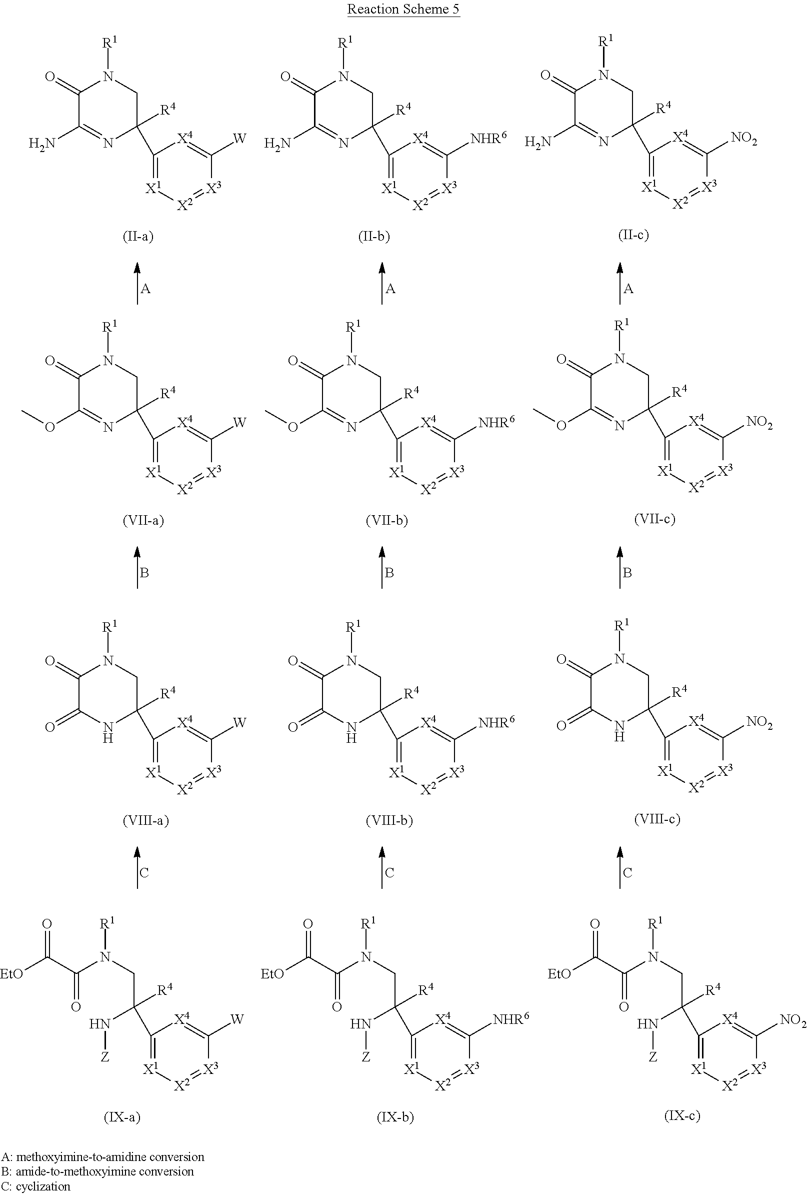

The intermediates according to Formula (II-a), (II-b) and (II-c) can be prepared from the corresponding intermediate compounds of Formula (VII-a), (VII-b) and (VII-c) by following art-known methoxyimine-to-amidine conversion procedures (reaction step A). Said conversion may be conducted by treatment of the said intermediate compounds of Formula (VII-a), (VII-b) and (VII-c) with an appropriate source of ammonia such as, for example, ammonium chloride, according to reaction scheme (5), a reaction that is performed in a suitable reaction-inert solvent, such as, for example, ethanol, under thermal conditions such as, for example, heating the reaction mixture at 75° C., for example for 18 hours.

Intermediate compounds of Formula (VII-a), (VII-b) and (VII-c) in the above reaction scheme (5) can be prepared from the corresponding intermediate compounds of Formula (VIII-a), (VIII-b) and (VIII-c) following art-known amide-to-methoxyimine conversion procedures (reaction step B). Said conversion may conveniently be conducted by treatment of the corresponding intermediate compounds of Formula (VIII-a), (VIII-b) and (VIII-c) with a methylating agent such as, for example, trimethyl-oxonium tetrafluoroborate, in a suitable reaction-inert solvent such as, for example, dichloromethane, at a moderately high temperature such as, for example, 25° C., for example for 3 days.

Intermediate compounds of Formula (VIII-a), (VIII-b) and (VIII-c) in the above reaction scheme (5) can be prepared from the corresponding intermediate compounds of Formula (IX-a), (IX-b) and (IX-c) following art-known known cyclization procedures (reaction step C). Said cyclization may conveniently be conducted by treatment of the said intermediate compounds of Formula (IX-a), (IX-b) and (IX-c) in an acidic medium such as, for example, hydrochloric acid in 1,4-dioxane, at a moderately high temperature such as, for example, 25° C., for example for 1 hour.

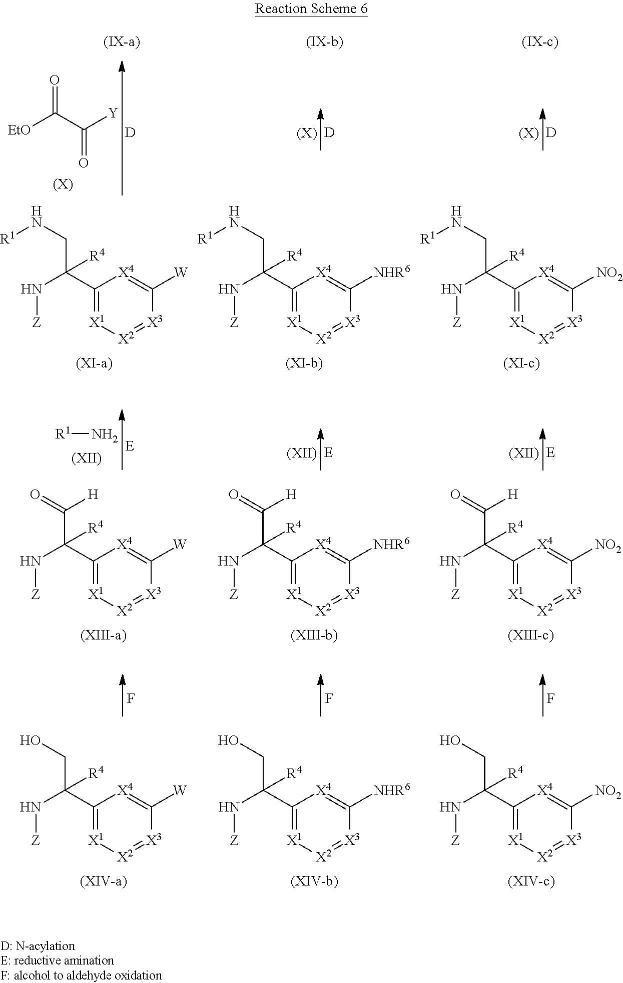

The intermediates according to Formula (IX-a), (IX-b) and (IX-c) in the above reaction scheme (6) can be prepared by reacting an intermediate compound of Formula (XI-a), (XI-b) and (XI-c) following art-known N-acylation procedures (reaction step D). Said N-acylation may conveniently be conducted by treatment of the corresponding intermediate compounds of Formula (XI-a), (XI-b) and (XI-c) with an intermediate compound of Formula (X) a reaction that is performed in a suitable reaction-inert solvent, such as, for example, dichloromethane, in the presence of a suitable base, such as, for example, triethylamine, at low temperature such as, for example, 0° C., for example for 3 hour. In reaction scheme (6), all variables are defined as in Formula (I) and halo is chloro or bromo.

The intermediates according to Formula (XI-a), (XI-b) and (XI-c) in the above reaction scheme (6) can be prepared by reacting an intermediate compound of Formula (XIII-a), (XIII-b) and (XIII-c) following art-known reductive amination procedures (reaction step E). Said reductive amination may conveniently be conducted by treatment of the corresponding intermediate compounds of Formula (XIII-a), (XIII-b) and (XIII-c) and (XIII-c) with an intermediate compound of Formula (XII) and a suitable reductive agent such as, for example, sodium triacetoxyborohydride, a reaction that is performed in a suitable reaction-inert solvent, such as, for example, dichloro-methane, in the presence of a suitable acidic catalyst such as, for example, acetic acid, at a moderately high temperature such as, for example, 25° C., for example for 5 hours. In reaction scheme (6), all variables are defined as in Formula (I).

The intermediates according to Formula (XIII-a), (XIII-b) and (XIII-c) in the above reaction scheme (6) can be prepared by reacting an intermediate compound of Formula (XIV-a), (XIV-b) and (XIV-c) following art-known alcohol to aldehyde oxidation procedures (reaction step F). Said oxidation may conveniently be conducted by treatment of the corresponding intermediate compounds of Formula (XIV-a), (XIV-b) and (XIV-c) with an oxidant agent such as, for example, the Dess-Martin periodinane [CAS: 87413-09-0], in a suitable reaction-inert solvent, such as, for example, dichloromethane, at low temperature such as, for example, 0° C., for example for 10 minutes and then at a moderately high temperature such as, for example, 25° C., for example for 1 hour. In reaction scheme (6), all variables are defined as in Formula (I).

The intermediates compounds of Formula (XIV-a), (XIV-b) and (XIV-c), wherein Z is a suitable N-protecting group such as, for example the tert-butoxy-carbonyl group, can generally be prepared following art-known Strecker type procedures.

### Pharmacology

The compounds of the present invention and the pharmaceutically acceptable compositions thereof inhibit BACE and therefore may be useful in the treatment or prevention of Alzheimer's Disease (AD), mild cognitive impairment (MCI), senility, dementia, dementia with Lewy bodies, cerebral amyloid angiopathy, multi-infarct dementia, Down's syndrome, dementia associated with Parkinson's disease and dementia associated with beta-amyloid.

The invention relates to a compound according to the general Formula (I), a stereoisomeric form thereof or a pharmaceutically acceptable acid or base addition salt or a solvate thereof, for use as a medicament.

The invention also relates to a compound according to the general Formula (I), a stereoisomeric form thereof or a the pharmaceutically acceptable acid or base addition salt or a solvate thereof, for use in the treatment or prevention of diseases or conditions selected from the group consisting of AD, MCI, senility, dementia, dementia with Lewy bodies, cerebral amyloid angiopathy, multi-infarct dementia, Down's syndrome, dementia associated with Parkinson's disease and dementia associated with beta-amyloid.

The invention also relates to the use of a compound according to the general Formula (I), a stereoisomeric form thereof or a pharmaceutically acceptable acid or base addition salt or a solvate thereof, for the manufacture of a medicament for the treatment or prevention of any one of the disease conditions mentioned hereinbefore.

In view of the utility of the compound of Formula (I), there is provided a method of treating warm-blooded animals, including humans, suffering from or a method of preventing warm-blooded animals, including humans, to suffer from any one of the diseases mentioned hereinbefore.

Said methods comprise the administration, i.e. the systemic or topical administration, preferably oral administration, of an effective amount of a compound of Formula (I), a stereoisomeric form thereof, a pharmaceutically acceptable addition salt or solvate thereof, to a warm-blooded animal, including a human.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent Alzheimer's disease or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agents in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

### Pharmaceutical Compositions

The present invention also provides compositions for preventing or treating diseases in which inhibition of beta-secretase is beneficial, such as Alzheimer's disease (AD), mild cognitive impairment, senility, dementia, dementia with Lewy bodies, Down's syndrome, dementia associated with stroke, dementia associated with Parkinson's disease and dementia associated with beta-amyloid. Said compositions comprise a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions: or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The following examples are intended to illustrate but not to limit the scope of the present invention.

### Experimental Part

Hereinafter, the term ‘m.p.” means melting point, ‘THF’ means tetrahydrofuran, ‘DMF’ means dimethylformamide, ‘DCM’ means dichloromethane, ‘AcOEt’ means ethyl acetate, “AcOH” means acetic acid, “MeOH” means methanol, DIPEA means “N,N-Diisopropylethylamine”, “rac” means racemic.

### A. Preparation of the Intermediates

### Example A1

### Preparation of rac-2-amino-2-(3-bromo-phenyl)-propionitrile

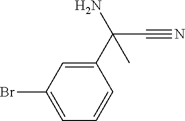

Trimethylsilylcyanide (20 g, 200 mmol) was added to a stirred solution of 3-bromo-acetophenone (20 g, 100 mmol) and NH₄Cl (11 g, 200 mmol) in NH₃/MeOH (400 mL). The mixture was stirred at room temperature for 4 days. Then the solvent was evaporated in vacuo and the residue was taken up in AcOEt (100 mL). The solid was filtered off and the filtrate was evaporated in vacuo to yield rac-2-amino-2-(3-bromo-phenyl)-propionitrile (20 g, 86% yield) that was used in the next step without further purification.

### Example A2

### Preparation of rac-2-amino-2-(3-bromo-phenyl)-propionic acid methyl ester

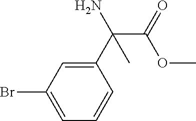

rac-2-Amino-2-(3-bromo-phenyl)-propionitrile (20 g, 88.9 mmol) was dissolved in HCl/MeOH (500 mL) and the mixture was refluxed for 4 days. After cooling to room temperature, AcOEt (100 mL) and water (100 mL) were added and after separation of the organic layer, the aqueous layer was washed with AcOEt (2×100 mL). The aqueous layer was then basified with aqueous ammonia solution until pH 8 and extracted with AcOEt (5×100 mL). The combined organic layers were dried (Na₂SO₄), filtered and the solvents evaporated in vacuo to yield rac-2-amino-2-(3-bromo-phenyl)-propionic acid methyl ester (10.6 g, 46% yield) as an oil.

### Example A3

### Preparation of rac-2-amino-2-(3-bromo-phenyl)-propan-1-ol

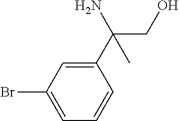

Lithium aluminium hydride (1 M in THF; 22 mL, 22 mmol) was added dropwise to a stirred solution of rac-2-amino-2-(3-bromo-phenyl)-propionic acid methyl ester (7.5 g, 29.1 mmol) in THF (200 ml) at −15° C. The mixture was left warming up slowly to 0° C. during 1 hour. Then more THF (150 ml) was added and sat. Na₂SO₄ was added dropwise until no more hydrogen was formed. Then anhydrous Na₂SO₄ was added and left stirring overnight at room temperature. The mixture was filtered over celite, rinsed with THF and the solvent evaporated in vacuo. The crude product was purified by flash column chromatography (silica gel; 7 M solution of ammonia in methanol in DCM 0/100 to 3/97). The desired fractions were collected and concentrated in vacuo to yield rac-2-amino-2-(3-bromo-phenyl)-propan-1-ol (5.70 g, 85% yield) as an oil.

### Example A4

### Preparation of rac-[1-(3-bromo-phenyl)-2-hydroxy-1-methyl-ethyl]-carbamic acid tert-butyl ester

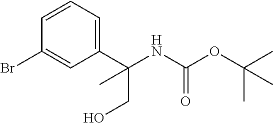 [SMILES: CC(C)(C)OC(=O)NC(C)(CO)c1cccc(Br)c1]

Di-tert-butyldicarbonate (4.84 g, 22.16 mmol) was added portion wise to a stirred solution of rac-2-amino-2-(3-bromo-phenyl)-propan-1-ol (1.7 g, 7.39 mmol) in a mixture of sat NaHCO₃ (15 mL) and THF (15 mL) at 0° C. The mixture was stirred at 0° C. for 10 minutes and at room temperature for 15 hours. The mixture was cooled in an ice water bath and acidified with stirring till pH 1-2 with KHSO₄. The organic layer was separated and the aqueous layer was further extracted with AcOEt. The combined organic layers were separated, dried (MgSO₄), filtered and the solvents evaporated in vacuo. The crude product was purified by flash column chromatography (silica; AcOEt in DCM 0/100 to 20/80). The desired fractions were collected and concentrated in vacuo to yield rac-[1-(3-bromo-phenyl)-2-hydroxy-1-methyl-ethyl]-carbamic acid tert-butyl ester (2.36 g, 93% yield) as colourless oil.

### Example A5

### Preparation of rac-[1-(3-bromo-phenyl)-1-methyl-2-oxo-ethyl]-carbamic acid tert-butyl ester

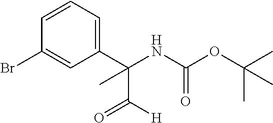 [SMILES: CC(C)(C)OC(=O)NC(C)(C=O)c1cccc(Br)c1]

Dess-Martin periodinane (3.55 g, 8.36 mmol) was added portionwise over 5 minutes to a solution of rac-[1-(3-bromo-phenyl)-2-hydroxy-1-methyl-ethyl]-carbamic acid tert-butyl ester (2.3 g, 6.97 mmol) in dry DCM at 0° C. The mixture was stirred at 0° C. for 10 minutes and at room temperature for 1 hour. The reaction mixture was quenched with NaHCO₃ (aqueous sat. soltn.) followed by NaHSO₃ (aqueous sat. soltn.). Then Et₂O was added and the mixture was stirred at room temperature for 30 minutes. The organic layer was separated and the aqueous layer was further extracted with Et₂O. The combined organic layers were separated, dried (MgSO₄), filtered and the solvents evaporated in vacuo. The crude product was purified by flash column chromatography (silica gel; DCM). The desired fractions were collected and concentrated in vacuo to yield rac-[1-(3-bromo-phenyl)-1-methyl-2-oxo-ethyl]-carbamic acid tert-butyl ester (2 g, 88% yield) as a colourless oil.

### Example A6

### Preparation of rac-[1-(3-bromo-phenyl)-1-methyl-2-methylamino-ethyl]-carbamic acid tert-butyl ester

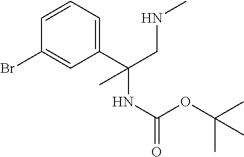 [SMILES: CNCC(C)(NC(=O)OC(C)(C)C)c1cccc(Br)c1]

Methylamine 2 M in THF (6.09 mL, 12.19 mmol) was added to a solution of rac-[1-(3-bromo-phenyl)-1-methyl-2-oxo-ethyl]-carbamic acid tert-butyl ester (2 g, 6.09 mmol) in a mixture of DCM (110 mL) and AcOH (2.01 mL). The mixture was stirred at room temperature for 1 hour. Then sodium triacetoxyborohydride (3.62 g, 17.06 mmol) was added and the mixture was stirred at room temperature for 5 hours. The mixture was diluted with DCM and poured into NaHCO₃ (aqueous sat. soltn.). The organic layer was separated and the aqueous layer was further extracted with DCM. The combined organic layers were separated, dried (MgSO₄), filtered and the solvents evaporated in vacuo to yield rac-[1-(3-bromo-phenyl)-1-methyl-2-methylamino-ethyl]-carbamic acid tert-butyl ester (2 g, 97% yield) that was used in the next step without further purification.

### Example A7

### Preparation of rac-N-[2-(3-bromo-phenyl)-2-tert-butoxycarbonylamino-propyl]-N-methyl-oxalamic acid ethyl ester

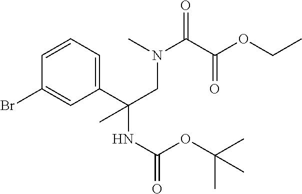 [SMILES: CCOC(=O)C(=O)N(C)CC(C)(NC(=O)OC(C)(C)C)c1cccc(Br)c1]

DIPEA (1.27 mL, 7.31 mmol) was added to a solution of rac-[1-(3-bromo-phenyl)-1-methyl-2-methylamino-ethyl]-carbamic acid tert-butyl ester (2.01 g, 6.09 mmol) in DCM (20 mL) and the mixture was cooled in an ice bath. Then ethyl oxalyl chloride (0.82 mL, 7.31 mmol) was added and the mixture was stirred at 0° C. for 3 hours. The mixture was diluted with NH₄Cl (aqueous sat. soltn.) and extracted with DCM. The organic layer was separated, dried (MgSO₄), filtered and the solvents evaporated in vacuo. The crude product was purified by flash column chromatography (silica gel; AcOEt in DCM 0/100 to 20/80). The desired fractions were collected and concentrated in vacuo to yield rac-N-[2-(3-bromo-phenyl)-2-tert-butoxycarbonylamino-propyl]-N-methyl-oxalamic acid ethyl ester (2.2 g, 81% yield) as a colourless oil.

### Example A8

### Preparation of rac-5-(3-bromo-phenyl)-1,5-dimethyl-piperazine-2,3-dione

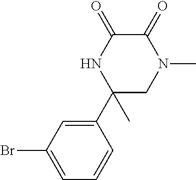 [SMILES: CN1CC(C)(c2cccc(Br)c2)NC(=O)C1=O]

Hydrochloric acid 4 M in dioxane (6.20 mL, 24.81 mmol) was added to rac-N-[2-(3-bromo-phenyl)-2-tert-butoxycarbonylamino-propyl]-N-methyl-oxalamic acid ethyl ester (2.2 g, 4.96 mmol) at room temperature. The mixture was stirred at room temperature for 1 hour. The solvent was evaporated in vacuo. The residue was suspended in DCM and washed with NaHCO₃ (aqueous sat. soltn.). The organic layer was separated, dried (MgSO₄), filtered and the solvents evaporated in vacuo to yield rac-5-(3-bromo-phenyl)-1,5-dimethyl-piperazine-2,3-dione (1.45 g, 98% yield) that was used in the next step without further purification.

### Example A9

### Preparation of rac-5-(3-bromo-phenyl)-3-methoxy-1,5-dimethyl-5,6-dihydro-1H-pyrazin-2-one

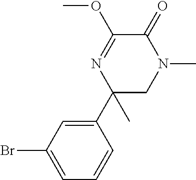 [SMILES: COC1=NC(C)(c2cccc(Br)c2)CN(C)C1=O]

Trimethyloxonium tetrafluoroborate (2.24 g, 15.14 mmol) was added to a solution of rac-5-(3-bromo-phenyl)-1,5-dimethyl-piperazine-2,3-dione (1.5 g, 5.05 mmol) in DCM (20 mL) and the mixture was stirred at room temperature for 3 days. Then the mixture was diluted with DCM and washed with cold NaHCO₃ (aqueous sat. soltn.). The organic layer was separated, dried (MgSO₄), filtered and the solvents evaporated in vacuo to yield rac-5-(3-bromo-phenyl)-3-methoxy-1,5-dimethyl-5,6-dihydro-1H-pyrazin-2-one (1.5 g, 95% yield) that was used in the next step without further purification.

### Example A10

### Preparation of rac-3-amino-5-(3-bromo-phenyl)-1,5-dimethyl-5,6-dihydro-1H-pyrazin-2-one

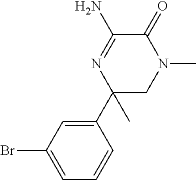 [SMILES: CN1CC(C)(c2cccc(Br)c2)N=C(N)C1=O]

Ammonium chloride (0.405 g, 7.57 mmol) was added to a solution of rac-5-(3-bromo-phenyl)-3-methoxy-1,5-dimethyl-5,6-dihydro-1H-pyrazin-2-one (1.57 g, 5.05 mmol) in EtOH (30 mL) and the mixture was stirred at 75° C. for 18 hours. The solvent was removed in vacuo and the residue was dissolved in DCM and washed with water. The organic layer was separated, dried (MgSO₄), filtered and the solvents evaporated in vacuo. The crude product was purified by flash column chromatography (silica gel; 7 M solution of ammonia in methanol in AcOEt 0/100 to 20/80). The desired fractions were collected and concentrated in vacuo to yield rac-3-amino-5-(3-bromo-phenyl)-1,5-dimethyl-5,6-dihydro-1H-pyrazin-2-one (0.75 g, 50% yield) as a white solid.

### Example A11

### Preparation of 5-methoxypyridine-3-boronic acid

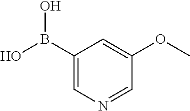

This compound was prepared by following the procedure described in WO 2005/037832.

### Example A12

### Preparation of rac-2-amino-2-(3-nitro-phenyl)-propionitrile

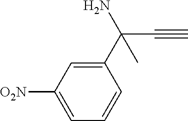

NH₄Cl (51.4 g, 969.7 mmol) was added to a stirred solution of 3-nitroacetophenone (80 g, 484.8 mmol) and trimethylsilylcyanide (96 g, 969.7 mmol) in NH₃/MeOH (800 mL). The mixture was stirred at room temperature for 2 days. Then the solvent was evaporated in vacuo and the residue was taken up in DCM. The solid was filtered off and the filtrate was evaporated in vacuo to yield rac-2-amino-2-(3-nitro-phenyl)-propionitrile (89 g, 96% yield) that was used in the next step without further purification.

### Example A13

### Preparation of rac-2-amino-2-(3-nitro-phenyl)-propionic acid methyl ester

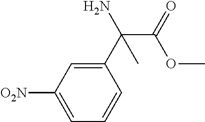 [SMILES: COC(=O)C(C)(N)c1cccc([N+](=O)[O-])c1]

rac-2-amino-2-(3-nitro-phenyl)-propionitrile (89 g, 465.5 mmol) was dissolved in HCl/MeOH (1000 mL) and the mixture was refluxed for 24 hours. The solvent was evaporated in vacuo and the residue was basified with NaHCO₃ (aqueous sat. soltn.) until pH 9 and extracted with AcOEt (3×100 mL). The combined organic layers were dried (Na₂SO₄), filtered and the solvents evaporated in vacuo to yield rac-2-amino-2-(3-nitro-phenyl)-propionic acid methyl ester (46 g, 44% yield).

### Example A14

### Preparation of rac-2-amino-2-(3-nitro-phenyl)-propan-1-ol

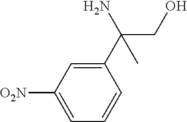

Sodium borohydride (10.2 g, 267.4 mmol) was added to a stirred solution of rac-2-amino-2-(3-nitro-phenyl)-propionic acid methyl ester (30 g, 133.7 mmol) in EtOH (200 ml). The mixture was stirred at room temperature for 3 hours. The solvent was evaporated in vacuo. Then water (200 mL) was added and the mixture was extracted with AcOEt (3×100 mL). The combined organic layers were dried (Na₂SO₄), filtered and the solvents evaporated in vacuo. The residue was dissolved in HCl/DCM (500 mL) and the mixture was stirred at room temperature for 1 hour. The mixture was filtered off and washed with 2-methoxy-2-methyl-propane to yield rac-2-amino-2-(3-nitro-phenyl)-propan-1-ol (27 g, 87% yield).

### Example A15

### Preparation of rac-[2-hydroxy-1-methyl-1-(3-nitro-phenyl)-ethyl]-carbamic acid tert-butyl ester

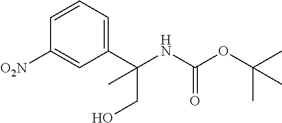 [SMILES: CC(C)(C)OC(=O)NC(C)(CO)c1cccc([N+](=O)[O-])c1]

Di-tert-butyldicarbonate (10.01 g, 45.87 mmol) was added portionwise to a stirred solution of rac-2-amino-2-(3-nitro-phenyl)-propan-1-ol (3 g, 15.29 mmol) in a mixture of NaHCO₃ (aqueous sat. soltn.) (30 mL) and THF (30 mL) at 0° C. The mixture was stirred at 0° C. for 10 minutes and at room temperature for 15 hours. The mixture was cooled in an ice water bath and acidified with stirring till pH 1-2 with KHSO₄. The organic layer was separated and the aqueous layer was further extracted with AcOEt. The combined organic layers were separated, dried (MgSO₄), filtered and the solvents evaporated in vacuo. The crude product was purified by flash column chromatography (silica gel; AcOEt in DCM 0/100 to 100/0). The desired fractions were collected and concentrated in vacuo to yield rac-[2-hydroxy-1-methyl-1-(3-nitro-phenyl)-ethyl]-carbamic acid tert-butyl ester (3.3 g, 73% yield) as a pale yellow oil that solidified upon standing.

### Example A16

### Preparation of rac-[1-methyl-1-(3-nitro-phenyl)-2-oxo-ethyl]-carbamic acid tert-butyl ester

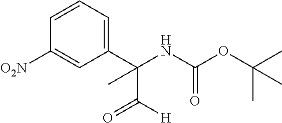 [SMILES: CC(C)(C)OC(=O)NC(C)(C=O)c1cccc([N+](=O)[O-])c1]

Dess-Martin periodinane (3.78 g, 8.91 mmol) was added portion wise over 5 minutes to a solution of rac-[2-hydroxy-1-methyl-1-(3-nitro-phenyl)-ethyl]-carbamic acid tert-butyl ester (2.2 g, 7.42 mmol) in dry DCM (35 mL) at 0° C. The mixture was stirred at 0° C. for 10 minutes and at room temperature for 1 hour. The reaction mixture was quenched with NaHCO₃ (aqueous sat. soltn.) followed by NaHSO₃ (aqueous sat. soltn.). Then Et₂O was added and the mixture was stirred at room temperature for 30 minutes. The organic layer was separated and the aqueous layer was further extracted with Et₂O. The combined organic layers were separated, dried (MgSO₄), filtered and the solvents evaporated in vacuo. The crude product was purified by flash column chromatography (silica gel; DCM). The desired fractions were collected and concentrated in vacuo to yield rac-[1-methyl-1-(3-nitro-phenyl)-2-oxo-ethyl]-carbamic acid tert-butyl ester (1.74 g, 80% yield) as a colourless oil that solidified upon standing.

### Example A17

### Preparation of rac-[1-methyl-2-methylamino-1-(3-nitro-phenyl)-ethyl]-carbamic acid tert-butyl ester

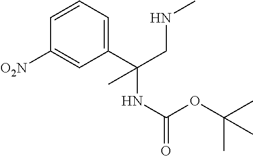 [SMILES: CNCC(C)(NC(=O)OC(C)(C)C)c1cccc([N+](=O)[O-])c1]

Methylamine 2 M in THF (5.78 mL, 11.55 mmol) was added to a solution of rac-[1-methyl-1-(3-nitro-phenyl)-2-oxo-ethyl]-carbamic acid tert-butyl ester (1.7 g, 5.78 mmol) in a mixture of DCM (100 mL) and AcOH (1.98 mL). The mixture was stirred at room temperature for 1 hour. Then sodium triacetoxyborohydride (3.43 g, 16.17 mmol) was added and the mixture was stirred at room temperature for 2 hours. The mixture was diluted with DCM and poured into NaHCO₃ (aqueous sat. soltn.). The organic layer was separated and the aqueous layer was further extracted with DCM. The combined organic layers were separated, dried (MgSO₄), filtered and the solvents evaporated in vacuo to yield rac-[1-methyl-2-methylamino-1-(3-nitro-phenyl)-ethyl]-carbamic acid tert-butyl ester (1.7 g, 95% yield) as a colourless oil that was used in the next step without further purification.

### Example A18

### Preparation of rac-N-[2-tert-butoxycarbonylamino-2-(3-nitro-phenyl)-propyl]-N-methyl-oxalamic acid ethyl ester

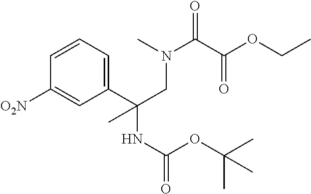 [SMILES: CCOC(=O)C(=O)N(C)CC(C)(NC(=O)OC(C)(C)C)c1cccc([N+](=O)[O-])c1]

DIPEA (1.15 mL, 6.59 mmol) was added to a solution of rac-[1-methyl-2-methyl-amino-1-(3-nitro-phenyl)-ethyl]-carbamic acid tert-butyl ester (1.7 g, 5.50 mmol) in DCM (20 mL) and the mixture was cooled in an ice bath. Then ethyl oxalyl chloride (0.74 mL, 6.59 mmol) was added and the mixture was stirred at 0° C. for 3 hours. The mixture was diluted with NH₄Cl (aqueous sat. soltn.) and extracted with DCM. The organic layer was separated, dried (MgSO₄), filtered and the solvents evaporated in vacuo. The crude product was purified by flash column chromatography (silica gel; AcOEt in DCM 0/100 to 20/80). The desired fractions were collected and concentrated in vacuo to yield rac-N-[2-tert-butoxycarbonylamino-2-(3-nitro-phenyl)-propyl]-N-methyl-oxalamic acid ethyl ester (2.2 g, 98% yield) as a colourless oil.

### Example A19

### Preparation of rac-1,5-dimethyl-5-(3-nitro-phenyl)-piperazine-2,3-dione

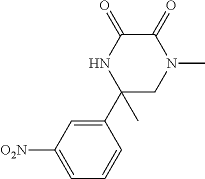 [SMILES: CN1CC(C)(c2cccc([N+](=O)[O-])c2)NC(=O)C1=O]

Hydrochloric acid 4 M in dioxane (6.88 mL, 27.5 mmol) was added to rac-N-[2-tert-butoxycarbonylamino-2-(3-nitro-phenyl)-propyl]-N-methyl-oxalamic acid ethyl ester (2.25 g, 5.5 mmol) at room temperature. The mixture was stirred at room temperature for 1 hour. The solvent was evaporated in vacuo. The residue was suspended in DCM and washed with NaHCO₃ (aqueous sat. soltn.). The organic layer was separated, dried (MgSO₄), filtered and the solvents evaporated in vacuo to yield rac-1,5-dimethyl-5-(3-nitro-phenyl)-piperazine-2,3-dione (1.2 g, 83% yield) that was used in the next step without further purification.

### Example A20

### Preparation of rac-3-methoxy-1,5-dimethyl-5-(3-nitro-phenyl)-5,6-dihydro-1H-pyrazin-2-one

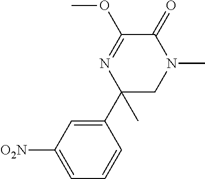 [SMILES: COC1=NC(C)(c2cccc([N+](=O)[O-])c2)CN(C)C1=O]

Trimethyloxonium tetrafluoroborate (2.02 g, 13.68 mmol) was added to a solution of rac-1,5-dimethyl-5-(3-nitro-phenyl)-piperazine-2,3-dione (1.2 g, 4.56 mmol) in DCM (10 mL) and the mixture was stirred at room temperature for 3 days. Then the mixture was diluted with DCM and washed with cold NaHCO3 (aqueous sat. soltn.). The organic layer was separated, dried (MgSO₄), filtered and the solvents evaporated in vacuo to yield rac-3-methoxy-1,5-dimethyl-5-(3-nitro-phenyl)-5,6-dihydro-1H-pyrazin-2-one (1 g, 79% yield) as a white solid that was used in the next step without further purification.

### Example A21

### Preparation of rac-3-amino-1,5-dimethyl-5-(3-nitro-phenyl)-5,6-dihydro-1H-pyrazin-2-one

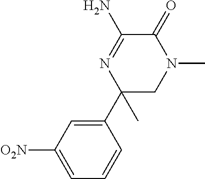 [SMILES: CN1CC(C)(c2cccc([N+](=O)[O-])c2)N=C(N)C1=O]

Ammonium chloride (0.19 g, 3.59 mmol) was added to a solution of rac-3-methoxy-1,5-dimethyl-5-(3-nitro-phenyl)-5,6-dihydro-1H-pyrazin-2-one (0.66 g, 2.39 mmol) in EtOH (30 mL) and the mixture was stirred at 75° C. for 18 hours. The solvent was removed in vacuo and the residue was dissolved in DCM and washed with water. The organic layer was separated, dried (MgSO₄), filtered and the solvents evaporated in vacuo. The crude product was purified by flash column chromatography (silica gel; 7 M solution of ammonia in methanol in AcOEt 0/100 to 20/80). The desired fractions were collected and concentrated in vacuo to yield rac-3-amino-1,5-dimethyl-5-(3-nitro-phenyl)-5,6-dihydro-1H-pyrazin-2-one (0.31 g, 49% yield) as a white solid.

### Example A22

### Preparation of rac-3-amino-5-(3-amino-phenyl)-1,5-dimethyl-5,6-dihydro-1H-pyrazin-2-one

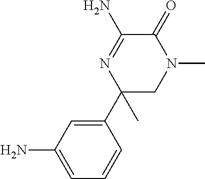 [SMILES: CN1CC(C)(c2cccc(N)c2)N=C(N)C1=O]

A solution of rac-3-amino-1,5-dimethyl-5-(3-nitro-phenyl)-5,6-dihydro-1H-pyrazin-2-one (0.31 g, 1.18 mmol) in a mixture of EtOH (28 mL) and AcOEt (15 mL) was hydrogenated in a H-Cube reactor (1 ml/min, 30 mm Pd/C₅% cartridge, full H₂ mode, room temperature, 2 cycles). The solvent was removed in vacuo to yield rac-3-amino-5-(3-amino-phenyl)-1,5-dimethyl-5,6-dihydro-1H-pyrazin-2-one (0.27 g, 98% yield) as a white solid that was used in the next step without further purification.

### Example A23

### Preparation of rac-2-amino-2-(5-bromo-2,4-difluoro-phenyl)-propionitrile

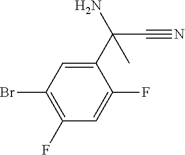

rac-2-amino-2-(5-bromo-2,4-difluoro-phenyl)-propionitrile was synthesized following a similar approach described in Example A1. Thus starting from commercially available 5-bromo-2,4-difluoroacetophenone (CAS Nr: 864773-64-8, 60 g, 255 mmol), rac-2-amino-2-(5-bromo-2,4-difluoro-phenyl)-propionitrile was obtained (31 g, 47% yield).

### Example A24

### Preparation of rac-2-amino-2-(5-bromo-2,4-difluoro-phenyl)-propionic acid

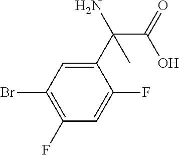

rac-2-amino-2-(5-bromo-2,4-difluoro-phenyl)-propionitrile (28 g, 107.6 mmol) was dissolved in 6 N HCl (300 mL) and acetic acid (300 mL) and the mixture was refluxed for 72 hours. After cooling to room temperature, the reaction mixture was concentrated in vacuo. AcOEt (400 mL) and water (300 mL) were added, the organic layer was separated, and the aqueous layer was washed with AcOEt (200 mL). The aqueous layer was neutralized to pH 7 and extracted with AcOEt (250 mL). The organic layer was dried (Na₂SO₄), filtered and the solvents evaporated in vacuo to yield rac-2-amino-2-(5-bromo-2,4-difluoro-phenyl)-propionic acid (22 g, 72% yield).

### Example A25

### Preparation of rac-2-amino-2-(5-bromo-2,4-difluoro-phenyl)-propionic acid methyl ester

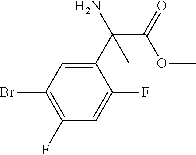 [SMILES: COC(=O)C(C)(N)c1cc(Br)c(F)cc1F]

rac-2-amino-2-(5-bromo-2,4-difluoro-phenyl)-propionic acid (22 g, 78.5 mmol) was dissolved in 4 N HCl/MeOH (400 mL) and the mixture was refluxed for 72 hours. After cooling to room temperature, the reaction mixture was concentrated in vacuo.

AcOEt (400 mL) and water (300 mL) were added and the aqueous layer was washed with AcOEt (200 mL). The aqueous layer was neutralized until pH 7 and extracted with AcOEt (250 mL). The organic layer was dried (Na₂SO₄), filtered and the solvents evaporated in vacuo to yield rac-2-amino-2-(5-bromo-2,4-difluoro-phenyl)-propionic acid methyl ester (20 g, 87% yield).

### Example A26

### Preparation of rac-2-amino-2-(5-bromo-2,4-difluoro-phenyl)-propan-1-ol

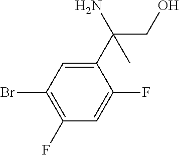

Sodium borohydride (4 g, 105 mmol) was added to a stirred solution of rac-2-amino-2-(5-bromo-2,4-difluoro-phenyl)-propionic acid methyl ester (20 g, 68 mmol) in ethanol (200 ml) and the mixture was stirred at 14° C. for 72 hours. Then the reaction mixture was concentrated in vacuo. AcOEt (500 mL) was added and the organic layer was washed with water, dried (Na₂SO₄), filtered and the solvents evaporated in vacuo to yield rac-2-amino-2-(5-bromo-2,4-difluoro-phenyl)-propan-1-ol (16 g, 88% yield).

### Example A27

### Preparation of rac-[1-(5-bromo-2,4-difluoro-phenyl)-2-hydroxy-1-methyl-ethyl]-carbamic acid tert-butyl ester

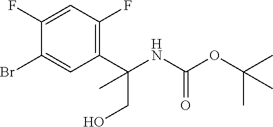 [SMILES: CC(C)(C)OC(=O)NC(C)(CO)c1cc(Br)c(F)cc1F]

rac-[1-(5-bromo-2,4-difluoro-phenyl)-2-hydroxy-1-methyl-ethyl]-carbamic acid tert-butyl ester was synthesized following a similar approach described in Example A4. Thus starting from rac-2-amino-2-(5-bromo-2,4-difluoro-phenyl)-propan-1-ol (4.2 g, 15.78 mmol), rac-[1-(5-bromo-2,4-difluoro-phenyl)-2-hydroxy-1-methyl-ethyl]-carbamic acid tert-butyl ester was obtained (5.3 g, 92% yield).

### Example A28

### Preparation of rac-[1-(5-bromo-2,4-difluoro-phenyl)-1-methyl-2-oxo-ethyl]-carbamic acid tert-butyl ester

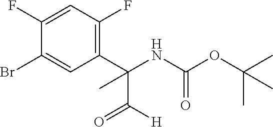 [SMILES: CC(C)(C)OC(=O)NC(C)(C=O)c1cc(Br)c(F)cc1F]

rac-[1-(5-bromo-2,4-difluoro-phenyl)-1-methyl-2-oxo-ethyl]-carbamic acid tert-butyl ester was synthesized following a similar approach described in Example A5. Thus starting from rac-[1-(5-bromo-2,4-difluoro-phenyl)-2-hydroxy-1-methyl-ethyl]-carbamic acid tert-butyl ester (3.2 g, 8.74 mmol), rac-[1-(5-bromo-2,4-difluoro-phenyl)-1-methyl-2-oxo-ethyl]-carbamic acid tert-butyl ester was obtained (3 g, 94% yield).

### Example A29

### Preparation of rac-[1-(5-bromo-2,4-difluoro-phenyl)-1-methyl-2-methylamino-ethyl]-carbamic acid tert-butyl ester

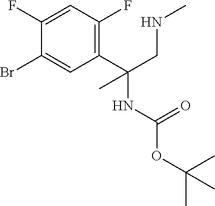 [SMILES: CNCC(C)(NC(=O)OC(C)(C)C)c1cc(Br)c(F)cc1F]

rac-[1-(5-bromo-2,4-difluoro-phenyl)-1-methyl-2-methylamino-ethyl]-carbamic acid tert-butyl ester was synthesized following a similar approach described in Example A6. Thus starting from rac-[1-(5-bromo-2,4-difluoro-phenyl)-1-methyl-2-oxo-ethyl]-carbamic acid tert-butyl ester (3 g, 8.24 mmol), rac-[1-(5-bromo-2,4-difluoro-phenyl)-1-methyl-2-methylamino-ethyl]-carbamic acid tert-butyl ester was obtained (1.8 g, 58% yield).

### Example A30

### Preparation of rac-N-[2-(5-bromo-2,4-difluoro-phenyl)-2-tert-butoxy-carbonylamino-propyl]-N-methyl-oxalamic acid ethyl ester

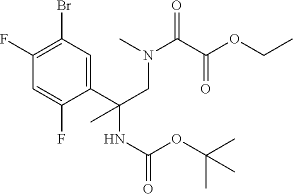 [SMILES: CCOC(=O)C(=O)N(C)CC(C)(NC(=O)OC(C)(C)C)c1cc(Br)c(F)cc1F]

rac-N-[2-(5-bromo-2,4-difluoro-phenyl)-2-tert-butoxycarbonylamino-propyl]-N-methyl-oxalamic acid ethyl ester was synthesized following a similar approach described in Example A7. Thus starting from rac-[1-(5-bromo-2,4-difluoro-phenyl)-1-methyl-2-methylamino-ethyl]-carbamic acid tert-butyl ester (1.8 g, 4.75 mmol), rac-N-[2-(5-bromo-2,4-difluoro-phenyl)-2-tert-butoxycarbonylamino-propyl]-N-methyl-oxalamic acid ethyl ester was obtained (1.9 g, 84% yield).

### Example A31

### Preparation of rac-5-(5-bromo-2,4-difluoro-phenyl)-1,5-dimethyl-piperazine-2,3-dione

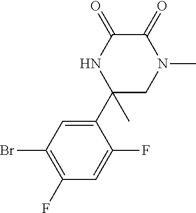 [SMILES: CN1CC(C)(c2cc(Br)c(F)cc2F)NC(=O)C1=O]

rac-5-(5-bromo-2,4-difluoro-phenyl)-1,5-dimethyl-piperazine-2,3-dione was synthesized following a similar approach described in Example A8. Thus starting from rac-N-[2-(5-bromo-2,4-difluoro-phenyl)-2-tert-butoxycarbonylamino-propyl]-N-methyl-oxalamic acid ethyl ester (1.9 g, 3.96 mmol), rac-5-(5-bromo-2,4-difluoro-phenyl)-1,5-dimethyl-piperazine-2,3-dione was obtained (1.1 g, 83% yield) and used as such in the next reaction.

### Example A32

### Preparation of rac-5-(5-bromo-2,4-difluoro-phenyl)-3-methoxy-1,5-dimethyl-5,6-dihydro-1H-pyrazin-2-one

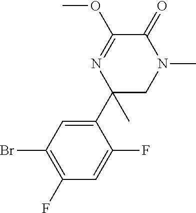 [SMILES: COC1=NC(C)(c2cc(Br)c(F)cc2F)CN(C)C1=O]

rac-5-(5-bromo-2,4-difluoro-phenyl)-3-methoxy-1,5-dimethyl-5,6-dihydro-1H-pyrazin-2-one was synthesized following a similar approach described in Example A9. Thus starting from rac-5-(5-bromo-2,4-difluoro-phenyl)-1,5-dimethyl-piperazine-2,3-dione (1 g, 3 mmol), rac-5-(5-bromo-2,4-difluoro-phenyl)-3-methoxy-1,5-dimethyl-5,6-dihydro-1H-pyrazin-2-one was obtained (1 g, 99% yield) and used as such in the next reaction.

### Example A33

### Preparation of rac-3-amino-5-(5-bromo-2,4-difluoro-phenyl)-1,5-dimethyl-5,6-dihydro-1H-pyrazin-2-one

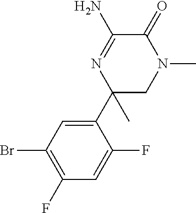 [SMILES: CN1CC(C)(c2cc(Br)c(F)cc2F)N=C(N)C1=O]

rac-3-amino-5-(5-bromo-2,4-difluoro-phenyl)-1,5-dimethyl-5,6-dihydro-1H-pyrazin-2-one was synthesized following a similar approach described in Example A9. Thus starting from rac-5-(5-bromo-2,4-difluoro-phenyl)-3-methoxy-1,5-dimethyl-5,6-dihydro-1H-pyrazin-2-one (1 g, 2.8 mmol), rac-3-amino-5-(5-bromo-2,4-difluoro-phenyl)-1,5-dimethyl-5,6-dihydro-1H-pyrazin-2-one was obtained (0.6 g, 63% yield).

### Example A34

### Preparation of rac-2-amino-2-(5-bromo-2-fluoro-phenyl)-propionitrile

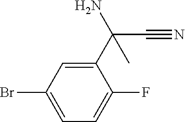

rac-2-amino-2-(5-bromo-2-fluoro-phenyl)-propionitrile was synthesized following a similar approach described in Example A1. Thus starting from commercially available 5-bromo-2-fluoroacetophenone (CAS Nr: 198477-89-3, 25 g, 115 mmol), rac-2-amino-2-(5-bromo-2-fluoro-phenyl)-propionitrile was obtained (28 g, quant. yield).

### Example A35

### Preparation of rac-2-amino-2-(5-bromo-2-fluoro-phenyl)-propionic acid

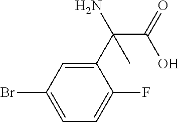

rac-2-amino-2-(5-bromo-2-fluoro-phenyl)-propionic acid was synthesized following a similar approach described in Example A24. Thus starting from rac-2-amino-2-(5-bromo-2-fluoro-phenyl)-propionitrile (27 g, 111 mmol), rac-2-amino-2-(5-bromo-2-fluoro-phenyl)-propionic acid was obtained (18 g, 62% yield).

### Example A36

### Preparation of rac-2-amino-2-(5-bromo-2-fluoro-phenyl)-propionic acid methyl ester

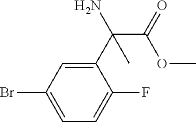

rac-2-amino-2-(5-bromo-2-fluoro-phenyl)-propionic acid (6 g, 22.9 mmol) was dissolved in H₂SO₄ (20 mL) and methanol (200 mL) and the mixture was refluxed for 48 hours. After cooling to room temperature, the reaction mixture was concentrated in vacuo. Water was added and the aqueous layer was basified with NaHCO₃ (aqueous sat. soltn.) until pH 8 and extracted with AcOEt. The combined organic layers were dried (MgSO₄), filtered and the solvents evaporated in vacuo to yield rac-2-amino-2-(5-bromo-2-fluoro-phenyl)-propionic acid methyl ester (6 g, 95% yield).

### Example A37

### Preparation of rac-2-amino-2-(5-bromo-2-fluoro-phenyl)-propan-1-ol

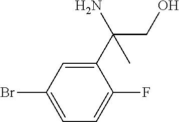

rac-2-amino-2-(5-bromo-2-fluoro-phenyl)-propan-1-ol was synthesized following a similar approach described in Example A26. Thus starting from rac-2-amino-2-(5-bromo-2-fluoro-phenyl)-propionic acid methyl ester (6 g, 21.7 mmol), rac-2-amino-2-(5-bromo-2-fluoro-phenyl)-propan-1-ol was obtained (5.2 g, 97% yield).

### Example A38

### Preparation of rac-[1-(5-bromo-2-fluoro-phenyl)-2-hydroxy-1-methyl-ethyl]-carbamic acid tert-butyl ester

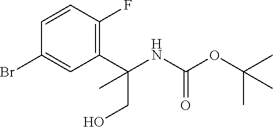 [SMILES: CC(C)(C)OC(=O)NC(C)(CO)c1cc(Br)ccc1F]

rac-[1-(5-bromo-2-fluoro-phenyl)-2-hydroxy-1-methyl-ethyl]-carbamic acid tert-butyl ester was synthesized following a similar approach described in Example A4. Thus starting from rac-2-amino-2-(5-bromo-2-fluoro-phenyl)-propan-1-ol (5.2 g, 20.96 mmol), rac-[1-(5-bromo-2-fluoro-phenyl)-2-hydroxy-1-methyl-ethyl]-carbamic acid tert-butyl ester was obtained (7.3 g, quant. yield).

### Example A39

### Preparation of rac-[1-(5-bromo-2-fluoro-phenyl)-1-methyl-2-oxo-ethyl]-carbamic acid tert-butyl ester

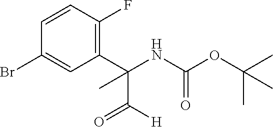 [SMILES: CC(C)(C)OC(=O)NC(C)(C=O)c1cc(Br)ccc1F]

rac-[1-(5-bromo-2-fluoro-phenyl)-1-methyl-2-oxo-ethyl]-carbamic acid tert-butyl ester was synthesized following a similar approach described in Example A5. Thus starting from rac-[1-(5-bromo-2-fluoro-phenyl)-2-hydroxy-1-methyl-ethyl]-carbamic acid tert-butyl ester (7.3 g, 20.96 mmol), rac-[1-(5-bromo-2-fluoro-phenyl)-1-methyl-2-oxo-ethyl]-carbamic acid tert-butyl ester was obtained (6 g, 83% yield).

### Example A40

### Preparation of rac-[1-(5-bromo-2-fluoro-phenyl)-1-methyl-2-methyl-amino-ethyl]-carbamic acid tert-butyl ester

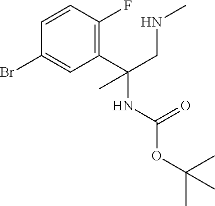 [SMILES: CNCC(C)(NC(=O)OC(C)(C)C)c1cc(Br)ccc1F]

rac-[1-(5-bromo-2-fluoro-phenyl)-1-methyl-2-methylamino-ethyl]-carbamic acid tert-butyl ester was synthesized following a similar approach described in Example A6. Thus starting from rac-[1-(5-bromo-2-fluoro-phenyl)-1-methyl-2-oxo-ethyl]-carbamic acid tert-butyl ester (6 g, 17.3 mmol), rac-[1-(5-bromo-2-fluoro-phenyl)-1-methyl-2-methylamino-ethyl]-carbamic acid tert-butyl ester was obtained (4 g, 64% yield).

### Example A41

### Preparation of rac-N-[2-(5-bromo-2-fluoro-phenyl)-2-tert-butoxy-carbonylamino-propyl]-N-methyl-oxalamic acid ethyl ester

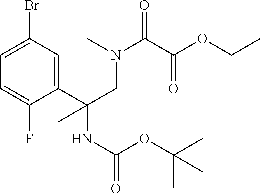 [SMILES: CCOC(=O)C(=O)N(C)CC(C)(NC(=O)OC(C)(C)C)c1cc(Br)ccc1F]

rac-N-[2-(5-bromo-2-fluoro-phenyl)-2-tert-butoxycarbonylamino-propyl]-N-methyl-oxalamic acid ethyl ester was synthesized following a similar approach described in Example A7. Thus starting from rac-[1-(5-bromo-2-fluoro-phenyl)-1-methyl-2-methylamino-ethyl]-carbamic acid tert-butyl ester (4 g, 11.07 mmol), rac-N-[2-(5-bromo-2-fluoro-phenyl)-2-tert-butoxycarbonylamino-propyl]-N-methyl-oxalamic acid ethyl ester was obtained (4.8 g, 93% yield) as a colourless oil.

### Example A42

### Preparation of rac-5-(5-bromo-2-fluoro-phenyl)-1,5-dimethyl-piperazine-2,3-dione

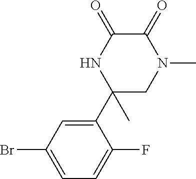 [SMILES: CN1CC(C)(c2cc(Br)ccc2F)NC(=O)C1=O]

rac-5-(5-bromo-2-fluoro-phenyl)-1,5-dimethyl-piperazine-2,3-dione was synthesized following a similar approach described in Example A8. Thus starting from rac-N-[2-(5-bromo-2-fluoro-phenyl)-2-tert-butoxycarbonylamino-propyl]-N-methyl-oxalamic acid ethyl ester (3.3 g, 7.15 mmol), rac-5-(5-bromo-2-fluoro-phenyl)-1,5-dimethyl-piperazine-2,3-dione was obtained (2.25 g, quant. yield) and used as such in the next reaction.

### Example A43

### Preparation of rac-5-(5-bromo-2-fluoro-phenyl)-3-methoxy-1,5-dimethyl-5,6-dihydro-1H-pyrazin-2-one

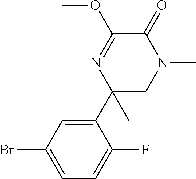 [SMILES: COC1=NC(C)(c2cc(Br)ccc2F)CN(C)C1=O]

rac-5-(5-bromo-2-fluoro-phenyl)-3-methoxy-1,5-dimethyl-5,6-dihydro-1H-pyrazin-2-one was synthesized following a similar approach described in Example A9. Thus starting from rac-5-(5-bromo-2-fluoro-phenyl)-1,5-dimethyl-piperazine-2,3-dione (3 g, 9.52 mmol), rac-5-(5-bromo-2-fluoro-phenyl)-3-methoxy-1,5-dimethyl-5,6-dihydro-1H-pyrazin-2-one was obtained (2.3 g, 73.4% yield) and used as such in the next reaction.

### Example A44

### Preparation of rac-3-amino-5-(5-bromo-2-fluoro-phenyl)-1,5-dimethyl-5,6-dihydro-1H-pyrazin-2-one

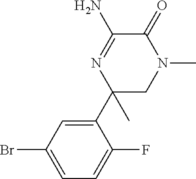 [SMILES: CN1CC(C)(c2cc(Br)ccc2F)N=C(N)C1=O]

rac-3-amino-5-(5-bromo-2-fluoro-phenyl)-1,5-dimethyl-5,6-dihydro-1H-pyrazin-2-one was synthesized following a similar approach described in Example A9. Thus starting from rac-5-(5-bromo-2-fluoro-phenyl)-3-methoxy-1,5-dimethyl-5,6-dihydro-1H-pyrazin-2-one (0.95 g, 2.89 mmol), rac-3-amino-5-(5-bromo-2-fluoro-phenyl)-1,5-dimethyl-5,6-dihydro-1H-pyrazin-2-one was obtained (0.5 g, 55% yield).

### Example A45

### Preparation of rac-3-amino-5-(5-amino-2-fluoro-phenyl)-1,5-dimethyl-5,6-dihydro-1H-pyrazin-2-one

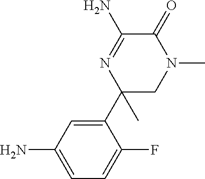 [SMILES: CN1CC(C)(c2cc(N)ccc2F)N=C(N)C1=O]

rac-3-amino-5-(5-bromo-2-fluoro-phenyl)-1,5-dimethyl-5,6-dihydro-1H-pyrazin-2-one (0.5 g, 1.59 mmol) was combined with NaN₃ (0.26 g, 3.98 mmol), CuI (0.379 g, 1.99 mmol) and Na₂CO₃ (0.337 g, 3.18 mmol) in DMSO (23 mL) and the reaction was degassed. After that, N,N′-dimethylethylenediamine (0.3 mL, 2.78 mmol) was added and the mixture was heated at 110° C. until completion of the reaction, about 1 hour. The reaction mixture was poured in DCM Ammonium hydroxide (28% in water) was added and the organic layer was separated and washed three times with ammonium hydroxide. Then organic layer was dried (Mg₂SO₄), filtered and concentrated in vacuo to yield rac-3-amino-5-(5-amino-2-fluoro-phenyl)-1,5-dimethyl-5,6-dihydro-1H-pyrazin-2-one (0.38 g, 95% yield).

### B. Preparation of the Final Compounds

### Example B1

### Preparation of rac-3′-(6-amino-2,4-dimethyl-5-oxo-2,3,4,5-tetrahydro-pyrazin-2-yl)-biphenyl-3-carbonitrile

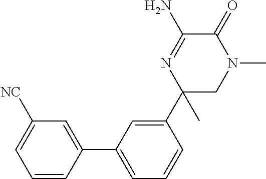 [SMILES: CN1CC(C)(c2cccc(-c3cccc(C#N)c3)c2)N=C(N)C1=O]

Tetrakis(triphenylphosphine)palladium (0) (0.015 g, 0.013 mmol) was added to a stirred suspension of rac-3-amino-5-(3-bromo-phenyl)-1,5-dimethyl-5,6-dihydro-1H-pyrazin-2-one (0.13 g, 0.439 mmol), (3-cyanophenyl)boronic acid (0.194 g, 1.317 mmol) and K₂CO₃ (0.182 g, 1.317 mmol) in 1,4-dioxane (4 mL) and EtOH (0.4 mL) at room temperature under nitrogen. The mixture was stirred at 150° C. for 20 minutes under microwave irradiation. The mixture was diluted with water and extracted with DCM. The organic layer was separated, dried (MgSO₄), filtered and the solvents evaporated in vacuo. The crude product was purified by flash column chromatography (silica gel; 7 M solution of ammonia in methanol in DCM 0/100 to 3/97). The desired fractions were collected and concentrated in vacuo to yield rac-3′-(6-amino-2,4-dimethyl-5-oxo-2,3,4,5-tetrahydro-pyrazin-2-yl)-biphenyl-3-carbonitrile (0.076 g, 54% yield) as an off-white solid.

### Example B2

### Preparation of rac-3-amino-5-[3-(5-methoxy-pyridin-3-yl)-phenyl]-1,5-dimethyl-5,6-dihydro-1H-pyrazin-2-one

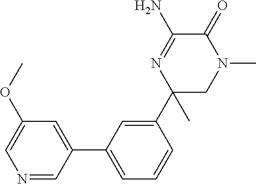 [SMILES: COc1cncc(-c2cccc(C3(C)CN(C)C(=O)C(N)=N3)c2)c1]

Tetrakis(triphenylphosphine)palladium (0) (0.012 g, 0.010 mmol) was added to a stirred suspension of rac-3-amino-5-(3-bromo-phenyl)-1,5-dimethyl-5,6-dihydro-1H-pyrazin-2-one (0.1 g, 0.338 mmol), 5-methoxypyridine-3-boronic acid (0.078 g, 0.506 mmol) and K₂CO₃ (0.140 g, 1.013 mmol) in 1,4-dioxane (5 mL) and EtOH (0.5 mL) at room temperature under nitrogen. The mixture was stirred at 150° C. for 30 minutes under microwave irradiation. The mixture was diluted with water and extracted with DCM. The organic layer was separated, dried (MgSO₄), filtered and the solvents evaporated in vacuo. The crude product was purified by flash column chromatography (silica gel; 7 M solution of ammonia in methanol in DCM 0/100 to 3/97). The desired fractions were collected and concentrated in vacuo to yield rac-3-amino-5-[3-(5-methoxy-pyridin-3-yl)-phenyl]-1,5-dimethyl-5,6-dihydro-1H-pyrazin-2-one (0.076 g, 69% yield) as an off-white solid.

### Example B3

### Preparation of rac-5-chloro-pyridine-2-carboxylic acid[3-(6-amino-2,4-dimethyl-5-oxo-2,3,4,5-tetrahydro-pyrazin-2-yl)-phenyl]-amide

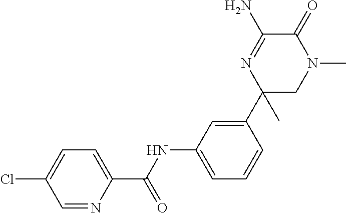 [SMILES: CN1CC(C)(c2cccc(NC(=O)c3ccc(Cl)cn3)c2)N=C(N)C1=O]

5-chloro-2-pyridinecarboxylic acid (0.071 g, 0.448 mmol) was added to a stirred solution of rac-3-amino-1,5-dimethyl-5-(3-nitro-phenyl)-5,6-dihydro-1H-pyrazin-2-one (0.080 g, 0.344 mmol) in DCM (5 mL) at room temperature. Then N,N-dimethylaniline (0.061 mL, 0.482 mmol) was added and after stirring at room temperature for 5 minutes 0-(7-azabenzotriazol-1-yl)-N,N,N′,N′-tetramethyluronium hexafluorophosphate (0.170 g, 0.448 mmol) was added. The mixture was stirred at room temperature for 3 hours. The mixture was diluted with Na₂CO₃ (aqueous sat. soltn.) and extracted with DCM. The organic layer was separated, dried (Na₂SO₄), filtered and the solvents evaporated in vacuo. The crude product was purified by flash column chromatography (silica gel; 7 M solution of ammonia in methanol in DCM 0/100 to 4/96). The desired fractions were collected and concentrated in vacuo to yield rac-5-chloro-pyridine-2-carboxylic acid[3-(6-amino-2,4-dimethyl-5-oxo-2,3,4,5-tetrahydro-pyrazin-2-yl)-phenyl]-amide (0.023 g, 18% yield) as a white solid.

### Example B4

### Preparation of (R)-5-chloro-pyridine-2-carboxylic acid[3-(6-amino-2,4-dimethyl-5-oxo-2,3,4,5-tetrahydro-pyrazin-2-yl)-phenyl]-amide and (S)-5-chloro-pyridine-2-carboxylic acid[3-(6-amino-2,4-dimethyl-5-oxo-2,3,4,5-tetrahydro-pyrazin-2-yl)-phenyl]-amide

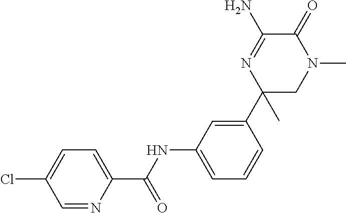 [SMILES: CN1CC(C)(c2cccc(NC(=O)c3ccc(Cl)cn3)c2)N=C(N)C1=O]

A sample of rac-5-chloro-pyridine-2-carboxylic acid[3-(6-amino-2,4-dimethyl-5-oxo-2,3,4,5-tetrahydro-pyrazin-2-yl)-phenyl]-amide (0.22 g) was purified by preparative SFC (Chiralpak® IC 250×20 mm, mobile phase 60% CO₂, 36% EtOH, 4% DCM and 0.3% iPrNH₂) to yield (R)-5-chloro-pyridine-2-carboxylic acid[3-(6-amino-2,4-dimethyl-5-oxo-2,3,4,5-tetrahydro-pyrazin-2-yl)-phenyl]-amide (0.064 g, 15%) and (S)-5-chloro-pyridine-2-carboxylic acid[3-(6-amino-2,4-dimethyl-5-oxo-2,3,4,5-tetrahydro-pyrazin-2-yl)-phenyl]-amide (0.065 g, 15%).

### Example B5

### Preparation of rac-5-amino-3-(2,4-difluoro-5-phenyl-phenyl)-1,3-dimethyl-2H-pyrazin-6-one

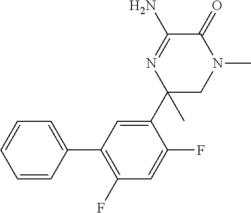 [SMILES: CN1CC(C)(c2cc(-c3ccccc3)c(F)cc2F)N=C(N)C1=O]

rac-5-amino-3-(2,4-difluoro-5-phenyl-phenyl)-1,3-dimethyl-2H-pyrazin-6-one was synthesized following a similar approach described in Example B2. Thus starting from rac-3-amino-5-(5-bromo-2,4-difluoro-phenyl)-1,5-dimethyl-5,6-dihydro-1H-pyrazin-2-one (0.1 g, 0.3 mmol), rac-5-amino-3-(2,4-difluoro-5-phenyl-phenyl)-1,3-dimethyl-2H-pyrazin-6-one was obtained as an oil. This oil was converted to the hydrochloric salt by addition of HCl (2 N in isopropanol) and crystallized from DIPE and acetonitrile to yield a white solid (0.048 g, 44% yield).

### Example B6

### Preparation of rac-5-amino-3-[2,4-difluoro-5-(5-methoxy-3-pyridyl)-phenyl]-1,3-dimethyl-2H-pyrazin-6-one, (R)-5-amino-3-[2,4-difluoro-5-(5-methoxy-3-pyridyl)phenyl]-1,3-dimethyl-2H-pyrazin-6-one and (S)-5-amino-3-[2,4-difluoro-5-(5-methoxy-3-pyridyl)phenyl]-1,3-dimethyl-2H-pyrazin-6-one

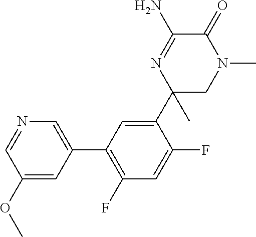 [SMILES: COc1cncc(-c2cc(C3(C)CN(C)C(=O)C(N)=N3)c(F)cc2F)c1]

rac-5-amino-3-[2,4-difluoro-5-(5-methoxy-3-pyridyl)phenyl]-1,3-dimethyl-2H-pyrazin-6-one was synthesized following a similar approach described in Example B2. Thus starting from rac-3-amino-5-(5-bromo-2,4-difluoro-phenyl)-1,5-dimethyl-5,6-dihydro-1H-pyrazin-2-one (0.55 g, 1.66 mmol), rac-5-amino-3-[2,4-difluoro-5-(5-methoxy-3-pyridyl)phenyl]-1,3-dimethyl-2H-pyrazin-6-one was obtained (0.4 g, 67% yield) as a solid.

A sample of rac-5-amino-3-[2,4-difluoro-5-(5-methoxy-3-pyridyl)phenyl]-1,3-dimethyl-2H-pyrazin-6-one (0.32 g) was then purified by preparative SFC (Chiralpak® Diacel AD 20×250 mm, mobile phase CO₂, MeOH with 0.2% iPrNH₂) to yield (R)-5-amino-3-[2,4-difluoro-5-(5-methoxy-3-pyridyl)phenyl]-1,3-dimethyl-2H-pyrazin-6-one (0.09 g, 15%) and (S)-5-amino-3-[2,4-difluoro-5-(5-methoxy-3-pyridyl)-phenyl]-1,3-dimethyl-2H-pyrazin-6-one (0.092 g, 15%).

### Example B7

### Preparation of rac-5-amino-3-(2,4-difluoro-5-pyrimidin-5-yl-phenyl)-1,3-dimethyl-2H-pyrazin-6-one, (R)-rac-5-amino-3-(2,4-difluoro-5-pyrimidin-5-yl-phenyl)-1,3-dimethyl-2H-pyrazin-6-one and (S)-rac-5-amino-3-(2,4-difluoro-5-pyrimidin-5-yl-phenyl)-1,3-dimethyl-2H-pyrazin-6-one

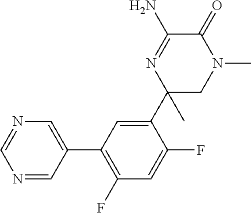 [SMILES: CN1CC(C)(c2cc(-c3cncnc3)c(F)cc2F)N=C(N)C1=O]

rac-5-amino-3-(2,4-difluoro-5-pyrimidin-5-yl-phenyl)-1,3-dimethyl-2H-pyrazin-6-one was synthesized following a similar approach described in Example B2. Thus starting from rac-3-amino-5-(5-bromo-2,4-difluoro-phenyl)-1,5-dimethyl-5,6-dihydro-1H-pyrazin-2-one (0.6 g, 1.81 mmol), rac-5-amino-3-(2,4-difluoro-5-pyrimidin-5-yl-phenyl)-1,3-dimethyl-2H-pyrazin-6-one was obtained (0.49 g, 82% yield) as a solid. This product was then purified by preparative SFC (Chiralpak® Diacel AD 20×250 mm, mobile phase CO₂, MeOH with 0.2% iPrNH₂) to yield (R)-5-amino-3-(2,4-difluoro-5-pyrimidin-5-yl-phenyl)-1,3-dimethyl-2H-pyrazin-6-one (0.098 g, 16%) and (S)-5-amino-3-(2,4-difluoro-5-pyrimidin-5-yl-phenyl)-1,3-dimethyl-2H-pyrazin-6-one (0.106 g, 18%).

### Example B8

### Preparation of (R)-5-chloro-pyridine-2-carboxylic acid[3-(6-amino-2,4-dimethyl-5-oxo-2,3,4,5-tetrahydro-pyrazin-2-yl)-4-fluoro-phenyl]-amide and (S)-5-chloro-pyridine-2-carboxylic acid[3-(6-amino-2,4-dimethyl-5-oxo-2,3,4,5-tetra-hydro-pyrazin-2-yl)-4-fluoro-phenyl]-amide

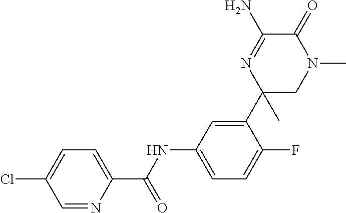 [SMILES: CN1CC(C)(c2cc(NC(=O)c3ccc(Cl)cn3)ccc2F)N=C(N)C1=O]

5-Chloro-2-pyridinecarboxylic acid (0.113 g, 0.72 mmol) was dissolved in MeOH (10 mL) and 4-(4,6-dimethoxy-1,3,5-triazin-2-yl)-4-methylmorpholinium chloride (0.53 g, 1.92 mmol) was added. After stirring the mixture for 5 minutes, a solution of rac-3-amino-5-(5-amino-2-fluoro-phenyl)-1,5-dimethyl-5,6-dihydro-1H-pyrazin-2-one (0.6 g, 1.81 mmol) in MeOH (5 mL) was added at 0° C., and the mixture was stirred for an additional 3 hours. After that, the reaction mixture was quenched with NaOH (1M in H₂O) at 0° C. and then extracted with EtOAc. The organic layer was washed with brine, then separated, dried (MgSO₄) and the solvent evaporated in vacuo. The crude material was purified by flash column chromatography (silica gel; 7 M solution of ammonia in methanol/DCM 0/100 to 5/95), the desired fractions were collected and the solvent evaporated in vacuo to afford rac-5-chloro-pyridine-2-carboxylic acid[3-(6-amino-2,4-dimethyl-5-oxo-2,3,4,5-tetrahydro-pyrazin-2-yl)-4-fluoro-phenyl]-amide (0.5 g). This product was then purified by preparative SFC (Chiralpak® Diacel OJ 20×250 mm, mobile phase CO₂, iPrOH with 0.2% iPrNH₂) to yield (R)-5-chloro-pyridine-2-carboxylic acid[3-(6-amino-2,4-dimethyl-5-oxo-2,3,4,5-tetrahydro-pyrazin-2-yl)-4-fluoro-phenyl]-amide (0.06 g, 9.6%) and (S)-5-chloro-pyridine-2-carboxylic acid[3-(6-amino-2,4-dimethyl-5-oxo-2,3,4,5-tetrahydro-pyrazin-2-yl)-4-fluoro-phenyl]-amide (0.065 g, 10%).

### Example B9

### Preparation of rac-5-amino-1,3-dimethyl-3-(3-phenyl-phenyl)-2H-pyrazin-6-one

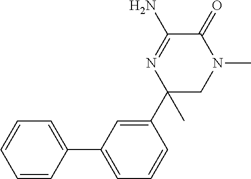 [SMILES: CN1CC(C)(c2cccc(-c3ccccc3)c2)N=C(N)C1=O]

rac-5-amino-1,3-dimethyl-3-(3-phenyl-phenyl)-2H-pyrazin-6-one was synthesized following a similar approach described in Example B2. Thus starting from rac-3-amino-5-(3-bromo-phenyl)-1,5-dimethyl-5,6-dihydro-1H-pyrazin-2-one (0.33 g, 1.11 mmol), rac-5-amino-1,3-dimethyl-3-(3-phenyl-phenyl)-2H-pyrazin-6-one was obtained as an oil. This oil was converted to the hydrochloric salt by addition of HCl (2 N in isopropanol) and crystallized from DIPE and acetonitrile to yield a white solid (0.072 g, 20% yield).

### Example B10

### Preparation of rac-5-amino-1,3-dimethyl-3-(3-pyrimidin-5-ylphenyl)-2H-pyrazin-6-one

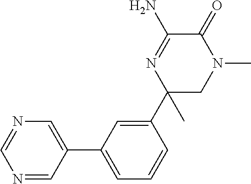 [SMILES: CN1CC(C)(c2cccc(-c3cncnc3)c2)N=C(N)C1=O]

rac-5-amino-1,3-dimethyl-3-(3-pyrimidin-5-ylphenyl)-2H-pyrazin-6-one was synthesized following a similar approach described in Example B2. Thus starting from rac-3-amino-5-(3-bromo-phenyl)-1,5-dimethyl-5,6-dihydro-1H-pyrazin-2-one (0.1 g, 0.34 mmol), rac-5-amino-1,3-dimethyl-3-(3-pyrimidin-5-ylphenyl)-2H-pyrazin-6-one was obtained as an off-white solid (0.075 g, 75% yield).

TABLE 1

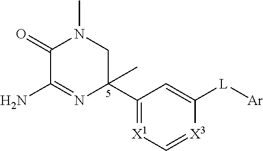

| Co. No. | Ex. No. | X¹ | X³ | 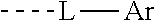 | C₅-stereochemistry |
|---|---|---|---|---|---|
| 1 | B1 | CH | CH | 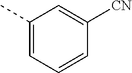 | RS |
| 2 | B2 | CH | CH | 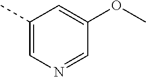 | RS |
| 3 | B3 | CH | CH | 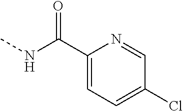 | RS |
| 4 | B4 | CH | CH | 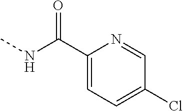 | S |
| 5 | B4 | CH | CH | 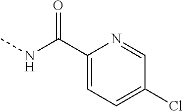 | R |
| 6 | B5 | CF | CF | 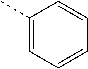 | RS |
| 7 | B6 | CF | CF | 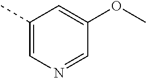 | RS |
| 8 | B6 | CF | CF | 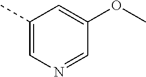 | S |
| 9 | B6 | CF | CF | 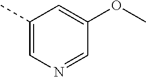 | R |
| 10 | B7 | CF | CF | 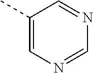 | RS |
| 11 | B7 | CF | CF | 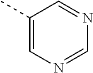 | S |
| 12 | B7 | CF | CF | 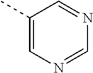 | R |
| 13 | B8 | CF | CH | 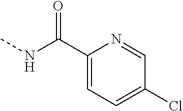 | S |
| 14 | B8 | CF | CH | 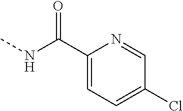 | R |
| 15 | B9 | CH | CH | 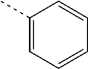 | RS |
| 16 | B10 | CH | CH | 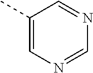 | RS |

### C. Analytical Part

### LCMS

For (LC)MS-characterization of the compounds of the present invention, the following methods were used.

### General Procedure A:

The HPLC measurement was performed using an HP 1100 (Agilent Technologies) system comprising a pump (quaternary or binary) with degasser, an autosampler, a column oven, a diode-array detector (DAD) and a column as specified in the respective methods. The MS detector was configured with either an electrospray ionization source or an ESCI dual ionization source (electrospray combined with atmospheric pressure chemical ionization). Nitrogen was used as the nebulizer gas. The source temperature was maintained either at 140° C. or 100° C. Data acquisition was performed either with MassLynx-Openlynx software or Chemsation-Agilent Data Browser software.

### General Procedure B:

The UPLC (Ultra Performance Liquid Chromatography) measurement was performed using an Acquity UPLC (Waters) system comprising a sampler organizer, a binary pump with degasser, a four column's oven, a diode-array detector (DAD) and a column as specified in the respective methods. The MS detector was configured with an ESCI dual ionization source (electrospray combined with atmospheric pressure chemical ionization). Nitrogen was used as the nebulizer gas. The source temperature was maintained at 140° C. Data acquisition was performed with MassLynx-Openlynx software.

### General Procedure C:

The HPLC measurement was performed using an Alliance HT 2795 (Waters) system comprising a quaternary pump with degasser, an autosampler, a diode-array detector (DAD) and a column as specified in the respective methods below, the column is hold at a temperature of 30° C. Flow from the column was split to a MS spectrometer. The MS detector was configured with an electrospray ionization source. The capillary needle voltage was 3 kV and the source temperature was maintained at 100° C. on the LCT (Time of Flight Zspray™ mass spectrometer from Waters). Nitrogen was used as the nebulizer gas. Data acquisition was performed with a Waters-Micromass MassLynx-Openlynx data system.

### General Procedure D:

The LC measurement was performed using an Acquity UPLC (Waters) system comprising a binary pump, a sample organizer, a column heater (set at 55° C.), a diode-array detector (DAD) and a column as specified in the respective methods below. Flow from the column was split to a MS spectrometer. The MS detector was configured with an electrospray ionization source. Mass spectra were acquired by scanning from 100 to 1000 in 0.18 seconds using a dwell time of 0.02 seconds. The capillary needle voltage was 3.5 kV and the source temperature was maintained at 140° C. Nitrogen was used as the nebulizer gas. Data acquisition was performed with a Waters-Micromass MassLynx-Openlynx data system.

### Method 1:

In addition to the general procedure A: Reversed phase HPLC was carried out on a Eclipse Plus-C18 column (3.5 μm, 2.1×30 mm) from Agilent, with a flow rate of 1.0 ml/min, at 60° C. The gradient conditions used are: 95% A (0.5 g/l ammonium acetate solution+5% acetonitrile), 5% B (acetonitrile) to 100% B in 5.0 minutes, kept till 5.15 minutes and equilibrated to initial conditions at 5.3 minutes until 7.0 minutes. Injection volume 2 μA High-resolution mass spectra (Time of Flight, TOF detector) were acquired by scanning from 100 to 750 in 0.5 seconds using a dwell time of 0.3 seconds. The capillary needle voltage was 2.5 kV for positive ionization mode and 2.9 kV for negative ionization mode. The cone voltage was 20 V for both positive and negative ionization modes. Leucine-Enkephaline was the standard substance used for the lock mass calibration.

### Method 2:

In addition to the general procedure B: Reversed phase UPLC was carried out on a BEH-C18 column (1.7 μm, 2.1×50 mm) from Waters, with a flow rate of 1.0 ml/min, at 50° C. without split to the MS detector. The gradient conditions used are: 95% A (0.5 g/l ammonium acetate solution+5% acetonitrile), 5% B (acetonitrile), to 40% A, 60% B in 3.8 minutes, to 5% A, 95% B in 4.6 minutes, kept till 5.0 minutes. Injection volume 2 μl. Low-resolution mass spectra (single quadrupole, SQD detector) were acquired by scanning from 100 to 1000 in 0.1 seconds using an inter-channel delay of 0.08 second. The capillary needle voltage was 3 kV. The cone voltage was 25 V for positive ionization mode and 30 V for negative ionization mode.

### Method 3:

In addition to the general procedure C: Reversed phase HPLC was carried out on a Waters Xterra-RP C18 column (3.5 μm, 4.6×100 mm) with a flow rate of 0.8 ml/min. Two mobile phases (mobile phase A: 100% 7 mM ammonium acetate; mobile phase B: 100% acetonitrile) were employed to run a gradient condition from 80% A and 20% B (hold for 0.5 minute) to 90% B in 4.5 minutes, 90% B for 4 minutes and reequilibrated with initial conditions for 3 minutes. An injection volume of 5 μl was used. Cone voltage was 20 V for positive and negative ionization mode. Mass spectra were acquired by scanning from 100 to 1000 in 0.4 seconds using an interscan delay of 0.3 seconds.

### Method 4:

In addition to the general procedure D: Reversed phase UPLC (Ultra Performance Liquid Chromatography) was carried out on a bridged ethylsiloxane/silica hybrid (BEH) C18 column (1.7 μm, 2.1×50 mm; Waters Acquity) with a flow rate of 0.8 ml/min. Two mobile phases (mobile phase A: 0.1% formic acid in H₂O/methanol 95/5; mobile phase B: methanol) were used to run a gradient condition from 95% A and 5% B to 5% A and 95% B in 1.3 minutes and hold for 0.2 minutes. An injection volume of 0.5 μl was used. Cone voltage was 10 V for positive ionization mode and 20 V for negative ionization mode.

### Method 5:

In addition to the general procedure D: Reversed phase UPLC (Ultra Performance Liquid Chromatography) was carried out on a bridged ethylsiloxane/silica hybrid (BEH) C18 column (1.7 μm, 2.1×50 mm; Waters Acquity) with a flow rate of 0.8 ml/min. Two mobile phases (25 mM ammonium acetate in H₂O/acetonitrile 95/5; mobile phase B: acetonitrile) were used to run a gradient condition from 95% A and 5% B to 5% A and 95% B in 1.3 minutes and hold for 0.3 minutes. An injection volume of 0.5 μl was used. Cone voltage was 30 V for positive ionization mode and 30 V for negative ionization mode.

### Melting Points

Values are either peak values or melt ranges, and are obtained with experimental uncertainties that are commonly associated with this analytical method.

### Mettler FP 81HT/FP90 Apparatus (Indicated by FP90 in Table 3)

For a number of compounds, melting points were determined in open capillary tubes on a Mettler FP81HT/FP90 apparatus. Melting points were measured with a temperature gradient of 1, 3, 5 or 10° C./minute. Maximum temperature was 300° C. The melting point was read from a digital display.

### DSC823e (Indicated by DSC in Table 3)

For a number of compounds, melting points were determined with a DSC823e (Mettler-Toledo). Melting points were measured with a temperature gradient of 30° C./minute. Maximum temperature was 400° C.

TABLE 2
Analytical data - Rₜ means retention time (in minutes),
[M + H]⁺ means the protonated mass of
the compound, method refers to the method used for (LC)MS.

| Co. Nr. | Rₜ | [M + H]⁺ | Method | Melting Point |
|---|---|---|---|---|
| 1 | 2.84 | 319 | 1 | 218.6° C. (FP90) |
| 2 | 1.26 | 325 | 2 | 230.4° C. (FP90) |
| 3 | 1.68 | 372 | 2 | n.d. |
| 4 | 4.92 | 372 | 3 | 218.8° C. (FP90) |
| 5 | 4.91 | 372 | 3 | 220.1° C. (FP90) |
| 6 | 0.98 | 330 | 4 | 141.5° C. (DSC) |
| 7 | 0.81 | 361 | 5 | 180.5° C. (DSC) |
| 8 | 0.80 | 361 | 5 | 173.6° C. (DSC) |
| 9 | 0.80 | 361 | 5 | 172.9° C. (DSC) |
| 10 | 0.68 | 332 | 5 | 145.2° C. (DSC) |
| 11 | 0.67 | 332 | 5 | 166.1° C. (DSC) |
| 12 | 0.67 | 332 | 5 | 166.2° C. (DSC) |
| 13 | 0.85 | 390 | 5 | 221.5° C. (DSC) |
| 14 | 0.85 | 390 | 5 | n.d. |
| 15 | n.d. | n.d. | n.d* | 227.3° C. (DSC) |
| 16 | 0.72 | 296 | 2 | 234.6° C. (FP90) |

n.d. means not determined
*LC/MS is available for another fraction (free base) not the salt.

### SFCMS

### General Procedure A:

The SFC measurement was performed using an Analytical SFC system from Berger instruments (Newark, Del., USA) comprising a FCM-1200 dual pump fluid control module for delivering carbon dioxide (CO₂) and modifier, a CTC Analytics automatic liquid sampler, a TCM-20000 thermal control module for column heating from room temperature to 80° C. An Agilent 1100 UV photodiode array detector equipped with a high-pressure flow cell standing up to 400 bars was used. Flow from the column was split to a MS spectrometer. The MS detector was configured with an atmospheric pressure ionization source. The following ionization parameters for the Waters ZQ mass spectrophotometer are: corona: 9 μa, source temp: 140° C., cone: 30 V, probe temp 450° C., extractor 3 V, desolvatation gas 400 L/hr, cone gas 70 L/hr. Nitrogen was used as the nebulizer gas. Data acquisition was performed with a Waters-Micromass MassLynx-Openlynx data system.

### General Procedure B:

The SFC measurement was performed using an Analytical SFC system from Berger Instruments (Newark, Del., USA) comprising a dual pump control module (FCM-1200) for delivery of carbon dioxide (CO₂) and modifier, a thermal control module for column heating (TCM2100) with temperature control in the range 1-150° C. and column selection valves (Valco, VICI, Houston, Tex., USA) for six different columns. The photodiode array detector (Agilent 1100, Waldbronn, Germany) is equipped with a high-pressure flow cell (up to 400 bar) and configured with a CTC LC Mini PAL auto sampler (Leap Technologies, Carrboro, N.C., USA). A ZQ mass spectrometer (Waters, Milford, Mass., USA) with an orthogonal Z-electrospray interface is coupled with the SFC-system. Instrument control, data collection and processing were performed with an integrated platform consisting of the SFC ProNTo software and Masslynx software.

### Method 1:

In addition to the general procedure A: The chiral separation in SFC was carried out on a CHIRALPAK IC DAICEL column (5 μm, 4.6×250 mm) at 35° C. with a flow rate of 3.0 ml/min. The mobile phase is CO₂, 60% Ethanol (containing 0.3% iPrNH₂) hold 11 min.

### Method 2:

In addition to the general procedure B: The chiral separation in SFC was carried out on a CHIRALPAK AD-H column (4.6×500 mm) at 50° C. with a flow rate of 3.0 ml/min. The mobile phase is CO₂, 20% MeOH (containing 0.2% iPrNH₂) hold 17.520 min, then from 20-50% MeOH/CO₂ at 10% rate and hold 4.20 min. at 50%.

### Method 3:

In addition to the general procedure B: The chiral separation in SFC was carried out on a CHIRALPAK AD-H column (4.6 mm×500 mm) at 50° C. with a flow rate of 3.0 ml/min. The mobile phase is CO₂, 20% MeOH (containing 0.2% iPrNH₂) hold 15.00 min, isocratic mode.

### Method 4:

In addition to the general procedure B: The chiral separation in SFC was carried out on a CHIRALCEL OJ-H column (4.6×500 mm) at 50° C. with a flow rate of 3.0 ml/min. The mobile phase is CO₂, 20% iPrOH (containing 0.2% iPrNH₂) hold 19.60 min, then from 20-50% iPrOH (containing 0.2% iPrNH₂) at 10% rate and hold 3.0 min at 50%.

TABLE 3
Analytical SFC data - Rₜ means retention time (in
minutes), [M + H]⁺ means the protonated
mass of the compound, method refers to the method
used for (SFC)MS analysis of enantiomerically pure compounds.

| | | | | | Isomer |
|---|---|---|---|---|---|
| Co. Nr. | Rₜ | [M + H]⁺ | UV Area % | Method | Elution Order |
| 5 | 6.10 | 372 | 100 | 1 | A |
| 4 | 8.29 | 372 | 100 | 1 | B |
| 9 | 10.77 | 361 | 100 | 2 | A |
| 8 | 13.10 | 361 | 100 | 2 | B |
| 12 | 6.67 | 332 | 99.1 | 3 | A |
| 11 | 8.03 | 332 | 100 | 3 | B |
| 14 | 8.15 | 390 | 100 | 4 | A |
| 13 | 9.73 | 390 | 100 | 4 | B |

### Optical Rotations

Optical rotations were measured on a Perkin-Elmer 341 polarimeter with a sodium lamp and reported as follows: [α]_{λ}^{t° C.} (c g/100 ml, solvent).

TABLE 4
Analytical data - Optical rotation values
for enantiomerically pure compounds

| | | Wavelength | Concentration | | Temp. |
|---|---|---|---|---|---|
| Co. Nr. | α_{D} (°) | (nm) | w/v % | Solvent | (° C.) |
| 5 | −81.1 | 589 | 0.54 | DMF | 20 |
| 4 | +87.2 | 589 | 0.50 | DMF | 20 |
| 9 | +47.1 | 589 | 0.37 | MeOH | 20 |
| 8 | −46.8 | 589 | 0.35 | MeOH | 20 |
| 12 | +23.4 | 589 | 0.36 | MeOH | 20 |
| 11 | −25.2 | 589 | 0.37 | MeOH | 20 |

### D. Pharmacological Examples

The compounds provided in the present invention are inhibitors of the β-site APP-cleaving enzyme 1 (BACE1) Inhibition of BACE1, an aspartic protease, is believed to be relevant for treatment of Alzheimer's Disease (AD). The production and accumulation of β-amyloid peptides (Aβ) from the β-amyloid precursor protein (APP) is believed to play a key role in the onset and progression of AD. Aβ is produced from the amyloid precursor protein (APP) by sequential cleavage at the N- and C-termini of the Aβ domain by β-secretase and γ-secretase, respectively.

Compounds of Formula (I) are expected to have their effect substantially at BACE1 by virtue of their ability to inhibit the enzymatic activity. The behaviour of such inhibitors tested using a biochemical Fluorescence Resonance Energy Transfer (FRET) based assay and a cellular αlisa assay in SKNBE2 cells described below and which are suitable for the identification of such compounds, and more particularly the compounds according to Formula (I), are shown in Table 3.

### Biochemical FRET Based Assay

This assay is a Fluorescence Resonance Energy Transfer Assay (FRET) based assay. The substrate for this assay is an APP derived 13 amino acids peptide that contains the ‘Swedish’ Lys-Met/Asn-Leu mutation of the amyloid precursor protein (APP) β-secretase cleavage site. This substrate also contains two fluorophores: (7-methoxy-coumarin-4-yl)acetic acid (Mca) is a fluorescent donor with excitation wavelength at 320 nm and emission at 405 nm and 2,4-Dinitrophenyl (Dnp) is a proprietary quencher acceptor. The distance between those two groups has been selected so that upon light excitation, the donor fluorescence energy is significantly quenched by the acceptor, through resonance energy transfer. Upon cleavage by BACE1, the fluorophore Mca is separated from the quenching group Dnp, restoring the full fluorescence yield of the donor. The increase in fluorescence is linearly related to the rate of proteolysis (Koike H et al. J Biochem. 1999, 126, 235-42).

Briefly in a 384-well format recombinant BACE1 protein in a final concentration of 1 μg/ml is incubated for 120 minutes at room temperature with 10 μm substrate in incubation buffer (40 mM Citrate buffer pH 5.0, 0.04% PEG, 4% DMSO) in the absence or presence of compound. Next the amount of proteolysis is directly measured by fluorescence measurement at T=0 and T=120 (excitation at 320 nm and emission at 405 nm). Results are expressed in RFU, as difference between T120 and T0.

A best-fit curve is fitted by a minimum sum of squares method to the plot of % Controlmin versus compound concentration. From this an 1050 value (inhibitory concentration causing 50% inhibition of activity) can be obtained.

$$\begin{matrix}LC = Median of the low control values \\ = Low control \text{:} Reaction without enzyme\end{matrix}$$

$$\begin{matrix}HC = Median of the High control values \\ = High Control \text{:} Reaction with enzyme\end{matrix}$$

$$\% Effect = 100 - [ ( sample - LC ) / ( HC - LC ) * 100 ]$$

$$\% Control = ( sample / HC ) * 100$$

$$\% Controlmin = ( sample - LC ) / ( HC - LC ) * 100$$

The following exemplified compounds were tested essentially as described above and exhibited the following the activity:

TABLE 5

| | Biochemical FRET |
|---|---|
| Co. Nr. | based assay pIC₅₀ |
| 1 | 4.56 |
| 2 | 4.81 |
| 3 | 6.34 |
| 4 | <4.52 |
| 5 | 6.53 |
| 6 | <4.52 |
| 7 | 5.12 |
| 8 | <4.52 |
| 9 | 5.42 |
| 10 | <4.52 |
| 11 | <4.52 |
| 12 | 5.04 |
| 13 | 4.54 |
| 14 | 7.40 |
| 15 | <4.52 |
| 16 | <4.52 |

Cellular αlisa Assay in SKNBE2 Cells

In two αlisa assays the levels of Aβtotal and Aβ42 produced and secreted into the medium of human neuroblastoma SKNBE2 cells are quantified. The assay is based on the human neuroblastoma SKNBE2 expressing the wild type Amyloid Precursor Protein (hAPP695). The compounds are diluted and added to these cells, incubated for 18 hours and then measurements of Aβ42 and Aβtotal are taken. Aβtotal and Aβ42 are measured by sandwich αlisa. αlisa is a sandwich assay using biotinylated antibody AbN/25 attached to streptavidin coated beads and antibody Ab4G8 or cAb42/26 conjugated acceptor beads for the detection of Aβtotal and Aβ42 respectively. In the presence of Aβtotal or Aβ42, the beads come into close proximity. The excitation of the Donor beads provokes the release of singlet oxygen molecules that triggers a cascade of energy transfer in the Acceptor beads, resulting in light emission. Light emission is measured after 1 hour incubation (excitation at 650 nm and emission at 615 nm).

A best-fit curve is fitted by a minimum sum of squares method to the plot of % Controlmin versus compound concentration. From this an IC50 value (inhibitory concentration causing 50% inhibition of activity) can be obtained.

$$\begin{matrix}LC = Median of the low control values \\ = Low control \text{:} cells preincubated without , \\ biotinylated Ab inthe α lisa\end{matrix}$$

$$\begin{matrix}HC = Median of the High control values \\ = High Control \text{:} cells preincubated without compound\end{matrix}$$

$$\% Effect = 100 - [ ( sample - LC ) / ( HC - LC ) * 100 ]$$

$$\% Control = ( sample / HC ) * 100$$

$$\% Controlmin = ( sample - LC ) / ( HC - LC ) * 100$$

The following exemplified compounds were tested essentially as described above and exhibited the following the activity:

TABLE 6

| | Cellular αlisa assay in | Cellular αlisa assay in |
|---|---|---|
| | SKNBE2 cells | SKNBE2 cells |
| Co. Nr. | Aβ42 pIC₅₀ | Aβtotal pIC₅₀ |
| 1 | 5.15 | 5.24 |
| 2 | 5.10 | 5.24 |
| 3 | 6.94 | 6.97 |
| 4 | <5 | <5 |
| 5 | 7.08 | 7.07 |
| 6 | <5 | <5 |
| 7 | 5.35 | 5.39 |
| 8 | <5 | <5 |
| 9 | 5.76 | 5.70 |
| 10 | <5 | <5 |
| 11 | <5 | <5 |
| 12 | 5.47 | 5.52 |
| 13 | <5 | <5 |
| 14 | 7.55 | 7.60 |
| 15 | <5 | <5 |
| 16 | <5 | <5 |

1. A compound of Formula (I) 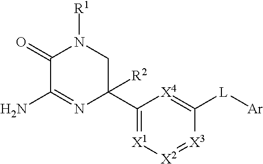
or a stereoisomeric form thereof, wherein
R¹ is selected from the group consisting of hydrogen, C₁₋₃alkyl, mono- and polyhalo-C₁₋₃alkyl, aryl and heteroaryl;
R² is selected from the group consisting of hydrogen, C₁₋₃alkyl, mono- and polyhalo-C₁₋₃alkyl, aryl and heteroaryl;
X¹, X², X³, X⁴ are independently C(R³) or N, provided that no more than two thereof rep-resent N; each R³ is selected from the group consisting of hydrogen, halo, C₁₋₃alkyl, mono- and polyhalo-C₁₋₃alkyl, cyano, C₁₋₃alkyloxy, mono- and polyhalo-C₁₋₃alkyloxy;
L is a bond or —N(R⁴)CO—, wherein R⁴ is hydrogen or C₁₋₃alkyl;
Ar is homoaryl or heteroaryl;
wherein homoaryl is phenyl or phenyl substituted with one, two or three substituents selected from the group consisting of halo, cyano, C₁₋₃alkyl, C₁₋₃alkyloxy, mono- and poly-halo-C₁₋₃alkyl;
heteroaryl is selected from the group consisting of pyridyl, pyrimidyl, pyrazyl, pyridazyl, furanyl, thienyl, pyrrolyl, pyrazolyl, imidazolyl, triazolyl, thiazolyl, thiadiazolyl, oxazolyl, and oxadiazolyl, each optionally substituted with one, two or three substituents selected from the group consisting of halo, cyano, C₁₋₃alkyl,
C₁₋₃alkyloxy, mono- and polyhalo-C₁₋₃alkyl; or an addition salt thereof.

2. The compound according to claim 1 wherein
R¹ and R² are independently selected from C₁₋₃alkyl;
X¹, X², X³, X⁴ are independently C(R³) wherein each R³ is selected from hydrogen and halo;
L is a bond or —N(R⁴)CO—, wherein R⁴ is hydrogen;
Ar is homoaryl or heteroaryl;
wherein homoaryl is phenyl or phenyl substituted with one or two substituents selected from the group consisting of halo, cyano, C₁₋₃alkyl, and C₁₋₃alkyloxy;
heteroaryl is selected from the group consisting of pyridyl, pyrimidyl, and pyrazyl, each optionally substituted with one or two substituents selected from the group consisting of halo, cyano, C₁₋₃alkyl, and C₁₋₃alkyloxy; or an addition salt thereof.

3. The compound according to claim 1 wherein
R¹ and R² are methyl;
X¹, X², X³, X⁴ are CH;
L is a bond or —N(R⁴)CO—, wherein R⁴ is hydrogen;
Ar is homoaryl or heteroaryl;
wherein homoaryl is phenyl or phenyl substituted with one or two substituents selected from chloro and cyano;
heteroaryl is selected from the group consisting of pyridyl, pyrimidyl, and pyrazyl, each optionally substituted with one or two substituents selected from the group consisting of chloro, fluoro, cyano, methyl, and methoxy; or an addition salt thereof.

4. The compound according to claim 1 wherein
R¹ and R² are methyl;
X¹ is CH or CF; X², X³ and X⁴ are CH;
L is —NHCO—;
Ar is 5-chloro-pyridin-2-yl; or an addition salt thereof.

5. The compound according to claim 1 wherein
R¹ and R² are methyl;
X¹ and X³ are CH or CF; X² and X⁴ are CH;
L is a bond;
Ar is 5-methoxy-pyridin-3-yl or pyrimidin-5-yl; or an addition salt thereof.

6. A pharmaceutical composition comprising a therapeutically effective amount of a compound as defined in claim 1 and a pharmaceutically acceptable carrier.

7. A process for preparing a pharmaceutical composition as defined in claim 1 wherein a pharmaceutically acceptable carrier is intimately mixed with a therapeutically effective amount of a compound as defined in any one of claims 1-5.

8. (canceled)

9. A method of treating a disorder selected from the group consisting of Alzheimer's disease, mild cognitive impairment, senility, dementia, dementia with Lewy bodies, Down's syndrome, dementia associated with stroke, dementia associated with Parkinson's disease and dementia associated with beta-amyloid, comprising administering to a subject in need of treatment for said disorder, a therapeutically effective amount of a compound as defined in claim 1.
